(19) United States
(12) Patent Application Publication
(10) Pub. No.: US 2023/0108025 A1
(43) Pub. Date: Apr. 6, 2023

(54) KIR 7.1 GENE THERAPY VECTORS AND METHODS OF USING THE SAME

(71) Applicant: Wisconsin Alumni Research Foundation, Madison, WI (US)

(72) Inventors: Bikash PATTNAIK, Middleton, WI (US); Pawan SHAHI, Madison, WI (US)

(21) Appl. No.: 17/911,367

(22) PCT Filed: Mar. 15, 2021
(86) PCT No.: PCT/US2021/022382

§ 371 Date: Sep. 13, 2022

Related U.S. Application Data
(60) Provisional application No. 62/989,215, filed on Mar. 13, 2020.

(51) Int. Cl. A61K 48/00; C12N 15/86

(52) U.S. Cl. CPC A61K 48/005; C12N 15/86; A61K 48/0058; C12N 2750/14143; C12N 2830/008; C12N 2830/48; C12N 2830/50

(57) ABSTRACT

The present invention is directed to improved AAV gene therapy constructs and pharmaceutical compositions for the expression of Kir7.1. The gene therapy constructs are particularly AAV vector comprising a promoter operably connected to a polynucleotide encoding a Kir7.1 polypeptide which is capable of being expressed in retinal pigment epithelium cells. Methods of treating a subject having a condition associated with insufficient expression or function of a Kir7.1 polypeptide are also provided.

### CROSS-REFERENCE TO RELATED APPLICATIONS

This application claims priority to U.S. Provisional Application No. 62/989,215 filed on Mar. 13, 2020, the contents of which are incorporated by reference in its entirety.

### STATEMENT REGARDING FEDERALLY SPONSORED RESEARCH

This invention was made with government support under EY024995 awarded by the National Institutes of Health. The government has certain rights in the invention.

### SEQUENCE LISTING

The content of the ASCII text file of the sequence listing named “960296_04122_ST25.txt” which is 62 kb in size was created on Mar. 11, 2021 and electronically submitted via EFS-Web herewith the application is incorporated herein by reference in its entirety.

### INTRODUCTION

Leber congenital amaurosis (LCA) is an inherited pediatric form of blindness characterized by severe loss of vision at birth. Children with LCA may also exhibit a variety of other abnormalities including roving eye movements (nystagmus), deep-set eyes, sensitivity to bright light, and central nervous system abnormalities. Typically, within an infant's first few months of life, parents notice a lack of visual responsiveness and nystagmus. Although the retinas of infants with LCA appear normal, little (if any) activity is detected in the retina by electroretinography (ERG). By early adolescence, however, various changes in the appearance of retina may be detected including pigmentary changes in the retinal pigment epithelium (RPE) and the presence of constricted blood vessels.

LCA is typically passed through families in an autosomal recessive pattern of inheritance. Mutations in at least 21 genes that are expressed in the outer retinal photoreceptors and retinal pigment epithelium (RPE) have been associated with LCA. Within the last decade, autosomal recessive mutations in the human KCNJ13 gene (603203 on chromosome locus 2q37.1) have been identified in patients with a specific form of LCA known as LCA16. To date, LCA16 pathogenic allelic variants include c.158G>A (p.Trp53Ter), c.359T>C (p.Iso120Thr), c.458C>T (p.Thr153Iso), c.496C>T (p.Arg166Ter), and c.722T>C (p.Leu241Pro). In addition, the compound heterozygous KCNJ113 mutations c.314 G>T (p.Ser105Iso) and c.655C>T (p.G219Ter) are known to cause early-onset retinal dystrophy in an LCA patient⁵. An autosomal dominant kcnj13 mutation, c.484C>T (p.Arg162Trp), causes early-onset blindness called snowflake vitreoretinal degeneration (SVD OMIM-193230).

The human KCNJ13 gene encodes an inward rectifying potassium channel—Kir7.1. The Kir7.1 protein is expressed in several human tissues including the cell apical processes of RPE, in which it modulates retinal function and health. The role of the Kir7.1 channel in other organs remains to be elucidated.

Although the role of Kir7.1 is beginning to be understood in conditions such as LCA16, there are no approved therapies to treat channelopathies or conditions associated with insufficient expression or function of the Kir7.1 protein. Accordingly, there is a need in the art for new therapies for treating such conditions.

### SUMMARY

In one aspect of the present invention, gene therapy vectors are provided. The gene therapy vectors are particularly an AAV vector comprising a promoter operably connected to a polynucleotide encoding a Kir7.1 polypeptide that allows for specific expression in retinal pigment epithelium cells.

In another aspect, the present invention relates to therapeutic compositions. The therapeutic compositions may include any of the gene therapy vectors described herein and a pharmaceutically-acceptable carrier.

In a further aspect of the present invention, methods of treating a subject having a condition associated with insufficient expression or function of a Kir7.1 polypeptide are provided. The methods may include administering a therapeutically effective amount of any one of the gene therapy vectors described herein or any one of the therapeutic compositions described herein to the subject.

### BRIEF DESCRIPTION OF THE DRAWINGS

FIGS. 1A-1E shows gene augmentation of W53X mutant expressing CHO cells had recovery of average inwardly rectifying K+ current (FIG. 9A. IV plot in red trace) compared to no current before (FIG. 9A. plot in black trace). (FIG. 9B) Average higher Rb+ current (red trace) in W53X mutant expressing cells after gene augmentation. (FIG. 9C) Net increase in Rb+ permeability increased (Blue) through Kir7.1 channel after gene augmentation. (FIG. 9D) Complete recovery of resting membrane potential (RMP) after AAV-Kir7.1 transduction of W53X expressing cells represented as blue box. (FIG. 9E) Western blot results showing expression of full length protein product after gene augmentation in lane W53X+AAV (red band).

FIG. 2 shows Kir7.1 gene-therapy in vivo. On the left is a control mouse showing normal wave form of electroretinogram and no change after gene augmentation. In the middle is a conditional knock out mice showing no c-wave in the right black trace. This wave which directly depends on Kir7.1 expression is completely recovered 4 weeks after gene therapy. Average result is shown in box plot with significant recovery of c-wave in experimental gene therapy.

FIGS. 3A-3E demonstrates results of a subset of mice that did not show c-wave recovery. (FIG. 3A) Graph representing the subset of mice that did not show c-wave recovery after injection of lentivirus carrying KCNJ13 gene driven by EF1a and VMD2 promoter. (FIG. 3B), (FIG. 3C) Optical coherence tomography (OCT) image of cKO mice with no c-wave during screening shows intact retina but wanes after 8 weeks revealing the progressive nature of retina degeneration over time due to the lack of Kir7.1 protein in RPE cell. (FIG. 3D), (FIG. 3E) OCT images showing the retinal structure from cKO mice those having the response from photoreceptors (a- and b-wave) but lacking c-wave response from RPE. Injection of the lentivirus carrying the KCNJ13 gene failed to restore c-wave, could be due to inefficiency of the RPE transduction or mutilation due to injection.

FIG. 4 depicts the expression of AAV27M8 in mature iPSC-RPE cells in culture, depicted by GFP expression.

FIG. 5 depicts that the AAV27M8 vector can be delivered by both intravitreal and subretinal routes to mouse RPE cells in the retina in vivo.

FIG. 6 demonstrates the viability of gene expression in the targeted RPE cells through intravitreal injection of AAV27M8 in the mouse model.

FIG. 7 depicts the plasmid map of the AAV-VMD2-Kir7.1 vector encoding Kir7.1 protein (i.e. KCNJ13). FIG. 8 depicts the sequence of the AAV-VMD2-Kir7.1 vector depicted in FIG. 7.

FIG. 9 is a graph depicting dose-dependent increase in expression of KCNJ13 using the AAV-VMD2-Kir7.1 vector in retinal pigment epithelium cells for 10⁸, 10⁹, and 10¹⁰ particles per cell.

FIG. 10 is a graph depicting dose -dependent increase in expression of AAV-ITR for 10⁸, 10⁹, and 10¹⁰ particles per cell.

### DETAILED DESCRIPTION

Here, the present inventors disclose an improved gene therapy vectors and therapeutic compositions that may be used to treat Leber Congenital Amaurosis 16 (LCA16) or other conditions associated with insufficient expression or function of a Kir7.1 protein. In the non-limiting Examples, the inventors surprisingly show that an AAV gene therapy approach may be used to effectively restore Kir7.1 polypeptide function in retinal pigment epithelium (RPE) cells either in vitro or in vivo resulting in RPE cells with rescued electrophysiological phenotypes. The inventors thus have discovered that gene therapy approaches may be used to effectively deliver the membrane protein Kir7.1. These results provide hope for potential curative therapeutics to treat Leber Congenital Amaurosis 16 (LCA16) or other conditions associated with insufficient expression or function of a Kir7.1 protein.

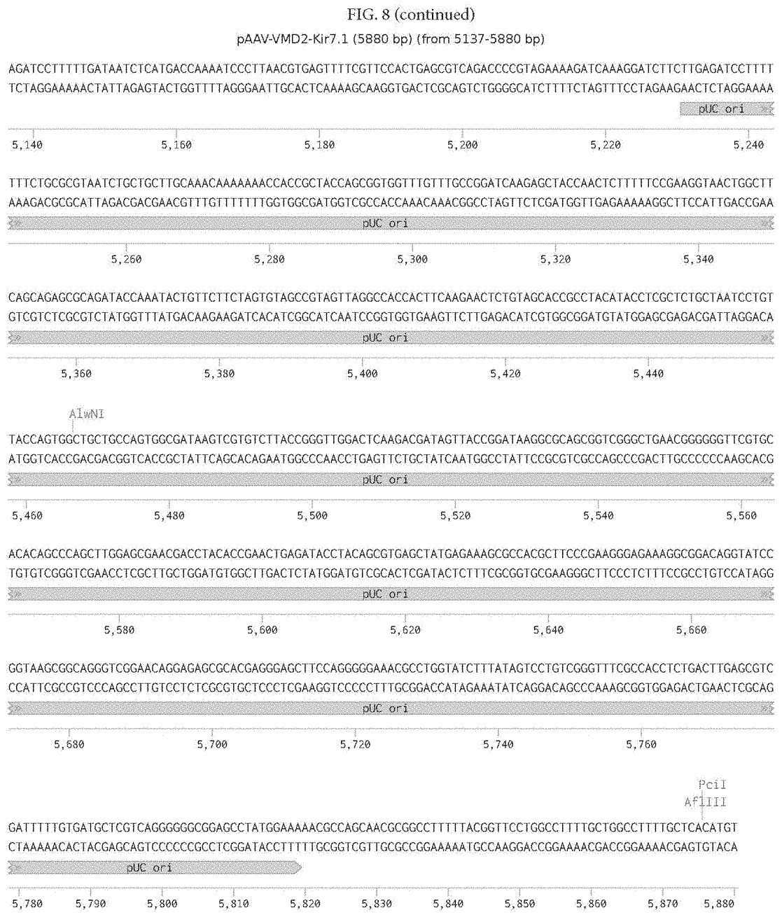

The inventors have surprisingly found an AAV gene therapy vector that encodes Kir7.1 and can specifically express Kir7.1 in RPE cells at sufficient levels to be used for correcting insufficient expression or function of Kir7.1 in conditions associated therewith. The AAV vectors described herein have advantages over the prior lentiviral vectors, providing RPE specific expression and high transduction efficiency by intra-vitreal and sub-retinal injection to allow for localized expression of the Kir7.1 protein. The specific viral vector is described more herein and contains the components depicted in FIG. 9, a plasmid map comprising in part the AAV vector, described more herein.

### Adeno-Associated Viral (AAV) Vectors

In one aspect of the present invention, AAV gene therapy vectors are provided. The gene therapy vectors may include a promoter operably connected to a polynucleotide encoding a Kir7.1 polypeptide. The general approach in certain aspects of the present invention is to provide a cell with an AAV virus particles encoding and capable of expressing a Kir7.1 polypeptide, thereby permitting the expression of the Kir7.1 polypeptide in the cell, particularly retinal pigment epithelium (RPE) cells. Following delivery of the AAV particles, the Kir7.1 polypeptide encoded by the AAV vector is synthesized by the transcriptional and translational machinery of the cell.

As used herein, the terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of natural or synthetic origin (which may be single-stranded or double-stranded and may represent the sense or the antisense strand). In some embodiments, the promoters and Kir7.1 polynucleotides or expression constructs encoding a Kir7.1 polypeptide described herein are encoded in double-stranded DNA, single-stranded DNA, or RNA.

As used herein, the terms “protein” or “polypeptide” or “peptide” may be used interchangeably to refer to a polymer of amino acids. A “polypeptide” as contemplated herein typically comprises a polymer of naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine).

As used herein, a “gene therapy vector” refers to viral or non-viral vector systems that may be used to deliver an expression construct encoding a Kir7.1 polypeptide into a cell (i.e., eukaryotic cell). There also are two primary approaches utilized in the delivery of an expression construct for the purposes of gene therapy; either indirect, ex vivo methods or direct, in vivo methods. Ex vivo gene transfer comprises vector modification of (host) cells in culture and the administration or transplantation of the vector modified cells to a gene therapy recipient. In vivo gene transfer comprises direct introduction of the vector (e.g., injection) into the target source or therapeutic gene recipient.

In certain embodiments of the invention, the expression construct encoding the Kir7.1 polypeptide may be stably or transiently maintained in the cell as a separate, episomal segment of DNA. Such nucleic acid segments or “episomes” encode sequences sufficient to permit maintenance and replication independent of or in synchronization with the host cell cycle. In some further limited embodiments, the expression construct is integrated into the host genome of the cell targeted. How the expression construct is delivered to a cell and/or where in the cell the nucleic acid remains is dependent on the type of vector employed. The following gene delivery methods provide the framework for choosing and developing the most appropriate gene delivery system for a preferred application.

In preferred embodiments, the gene therapy vector is a viral vector, particularly an adeno-associated virus (AAV) vector. The viral vector may be a virus particle or may be encoded on a DNA plasmid. The capacity of certain viral vectors to efficiently infect or enter cells, to integrate into a host cell genome and stably express viral genes, have led to the development and application of a number of different viral vector systems (Robbins et al., 1998). Viral systems are currently being developed for use as vectors for ex vivo and in vivo gene transfer. For example, adenovirus, herpes-simplex virus, retrovirus and adeno-associated virus vectors are being evaluated currently for treatment of human diseases.

The preferred viral vector of the present invention is an adeno-associated viral (AAV) vectors.

Here, in the non-limiting Examples, the present inventors demonstrate that a polynucleotide encoding a Kir7.1 polypeptide could successfully be introduced and expressed in retinal pigment epithelium (RPE) cells either in vitro or in vivo using an adeno-associated viral (AAV) vector which showed good expression and ability to target RPE cells in vivo so as to rescue functional defects in a kcnj13 gene. Accordingly, in some embodiments, the viral vector may be an AAV, suitably an AAV2 vector.

Adeno-associated virus (AAV), a member of the parvovirus family, is a human non-enveloped virus that is increasingly being used for gene delivery therapeutics. AAV has several advantageous features not found in other viral systems. First, AAV can infect a wide range of host cells, including non-dividing cells. Second, AAV can infect cells from different species. Third, AAV has not been associated with any human or animal disease and does not appear to alter the biological properties of the host cell upon integration. For example, it is estimated that 80-85% of the human population has been exposed to AAV. Finally, AAV is stable at a wide range of physical and chemical conditions which lends itself to production, storage and transportation requirements.

The AAV genome is a linear, single-stranded DNA molecule containing 4681 nucleotides. The AAV genome generally comprises an internal non-repeating genome flanked on each end by inverted terminal repeats (ITRs) of approximately 145 bp in length. The ITRs have multiple functions, including origins of DNA replication, and as packaging signals for the viral genome. AAV ITRs may be derived from any of several AAV serotypes, including AAV1, AAV2, AAV3, AAV4, AAVS, AAV6, AAV7, AAV8, AAV9, AAV10, AAV11, avian AAVs, bovine AAVs etc. The 5′ and 3′ ITRs of the AAV viral vectors disclosed herein may be derived from any of these AAV serotypes. Thus, rAAV vector design and production allow for exchanging the capsid proteins between different AAV serotypes. Homologous vectors comprising an expression cassette flanked by e.g., AAV2-ITRs and packaged in an AAV2 capsid, can be produced as well as heterologous, hybrid vectors where the transgene expression cassette is flanked by e.g., AAV2 ITRs, but the capsid originates from another AAV serotype such as AAVS for example. Suitably, in some embodiments, the present inventors have found that AAV2 viral vectors may be used to effectively deliver Kir7.1 expression constructs into cells.

The internal non-repeated portion of the AAV genome includes two large open reading frames, known as the AAV replication (rep) and capsid (cap) genes. The rep and cap genes code for viral proteins that allow the virus to replicate and package the viral genome into a virion. A family of at least four viral proteins is expressed from the AAV rep region, Rep 78, Rep 68, Rep 52, and Rep 40, named according to their apparent molecular weight. The AAV cap region encodes at least three proteins, VP1, VP2, and VP3.

AAV is a helper-dependent virus requiring co-infection with a helper virus (e.g., adenovirus, herpesvirus or vaccinia) in order to form AAV virions. In the absence of co-infection with a helper virus, AAV establishes a latent state in which the viral genome inserts into a host cell chromosome, but infectious virions are not produced. Subsequent infection by a helper virus “rescues” the integrated genome, allowing it to replicate and package its genome into infectious AAV virions. Although AAV can infect cells from different species, the helper virus must be of the same species as the host cell (e.g., human AAV will replicate in canine cells co-infected with a canine adenovirus).

AAV has been engineered to deliver genes of interest by deleting the internal non-repeating portion of the AAV genome and inserting a heterologous gene between the ITRs. The heterologous gene may be functionally linked to a heterologous promoter (constitutive, cell-specific, or inducible) capable of driving gene expression in target cells. To produce infectious recombinant AAV (rAAV) containing a heterologous gene, a suitable producer cell line is transfected with a rAAV vector containing a heterologous gene. The producer cell is concurrently transfected with a second plasmid harboring the AAV rep and cap genes under the control of their respective endogenous promoters or heterologous promoters. Finally, the producer cell is infected with a helper virus. Once these factors come together, the heterologous gene is replicated and packaged as though it were a wild-type AAV genome. When target cells are infected with the resulting rAAV virions, the heterologous gene enters and is expressed in the target cells. Because the target cells lack the rep and cap genes and the adenovirus helper genes, the rAAV cannot further replicate, package or form wild-type AAV.

Suitable AAV vectors are known in the art. For example, suitable AAV vectors include AAV2/5, demonstrated in “AAV2/5-mediated gene therapy in iPSC-derived retinal pigment epithelium of a choroideremia patient”, incorporated by reference in its entirety. See, e.g., Cereso et. al. Mol Ther Methods Clin Dev. 2014. Further examples of AAV vectors that can suitably be adapted for the present gene delivery can be found in “Comparative AAV-eGFP Transgene Expression Using Vector Serotypes 1-9, 7m8, and 8b in Human Pluripotent Stem Cells, RPEs, and Human and Rat Cortical Neurons.” See Duong et.al. Stem Cells Int. 2019.

In one example, the AAV gene therapy vector is an AAV7m8 vector (See, e.g., Dalkara et al., In vivo-directed evolution of a new adeno-associated virus for therapeutic outer retinal gene therapy from the vitreous, Science Translational Medicine, 12 Jun. 2013, Vol. 5, Issue 189ra76, incorporated by reference) comprising the Kir7.0 (kcnj13) gene and capable of expressing KCNJ13 (Kir7.1).

Specifically, in one embodiment, the AAV vector is a polynucleotide of SEQ ID NO:22 or a polynucleotide having at least 90% sequence identity to SEQ ID NO:22, alternatively at least 95% sequence identity to SEQ ID NO:22, alternatively at least 98% sequence identity to SEQ ID NO:22, alternatively at least 99% sequence identity to SEQ ID NO:22, alternatively 100% identity to SEQ ID NO:22. The AAV vector suitably comprises a promoter active in the retinal pigment epithelium (RPE) in the eye of a subject, and in some embodiments, a promoter that is specifically active in RPE cells as opposed to other cells. As described in the Examples, this AAV vector may be administered intravitreally or subretinaly to the subject to express the KCNJ13 in RPE cells.

In one embodiment, the disclosure provides a adeno-associated viral (AAV) gene therapy vector comprising: a) a 5′ ITR; b) a retinal pigment epithelium (RPE) specific promoter, c) a polynucleotide sequence encoding KCNJ13 protein or a polynucleotide having at least 90% sequence identity to KCNJ13; d) a posttranscriptional regulatory element (PRE), e) a polyadenylation signal, and 0 a 3′ITR (AAV 3′ inverted terminal repeat).

In one embodiment, the adeno-associated viral (AAV) gene therapy vector comprises: a) a 5′ ITR comprising SEQ ID NO:23; b) a retinal pigment epithelium (RPE) specific promoter , c) a polynucleotide sequence of SEQ ID NO:25 encoding KCNJ13 protein or a polynucleotide having at least 90% sequence identity to SEQ ID NO:25; d) a posttranscriptional regulatory element (PRE), e) a polyadenylation signal, and f) a 3′ITR (AV 3′ inverted terminal repeat) of SEQ ID NO:28. In further examples, the RPE specific promoter is VMD2 comprising SEQ ID NO:24 or a polynucleotide sequence having at least 90% sequence similarity to SEQ ID NO:24. RPE specific promoter allows for the targeted expression of the Kir7.1 into RPE cells over other cells found in the retina. In additional examples, the posttranscriptional regulatory element of d) is a woodchuck PRE comprising SEQ ID NO:26. In further embodiments, the AAV gene therapy vector comprises the polyadenylation signal of (e) of SEQ ID NO:27.

A suitable example of an AAV vector of the present invention can be found in SEQ ID NO:31 or a polynucleotide sequence having at least 90% sequence identity to SEQ ID NO:31 and encoding KCNJ13 protein that is able to be expressed in the target cell.

In another embodiment, the disclosure provides a construct comprising the AAV gene therapy vector described herein. As used herein, the term “construct” or “nucleic acid construct” refers to is an artificially designed nucleic acid molecule. Nucleic acid constructs may be part of a vector that is used, for example, to transform a cell. When referring to a nucleic acid molecule alone (as opposed to a viral particle, see below), the term “vector” or “plasmid” is used herein to refer to a nucleic acid molecule capable of propagating another nucleic acid to which it is linked. The term includes the vector as a self-replicating nucleic acid structure that can be packaged into viral particles and can be expressed in dividing and non-dividing cells either extrachromosomally or integrated into the host cell genome. Certain vectors are capable of directing the expression of nucleic acids to which they are operatively linked. Such vectors are referred to as “expression vectors”.

The construct described herein may be in preferred embodiments a plasmid capable of propagation in bacteria. In some embodiments, the construct comprising an antibiotic resistance gene and an origin of replication to allow for growth in bacteria.

In some embodiments, the AAV vector or constructs consists of components (a)-(f). By “consist of” we mean that a constructs of the present invention will consist of components (a)-(f) and possibly other regulatory elements necessary for construct function. For example, the constructs may include additional sequences to facilitate the addition or removal of functional elements, such as restriction sites, or sequences necessary for the replication of the construct itself but do not alter the function expression of Kir7.1 from the construct, but do not change the overall function of the construct.

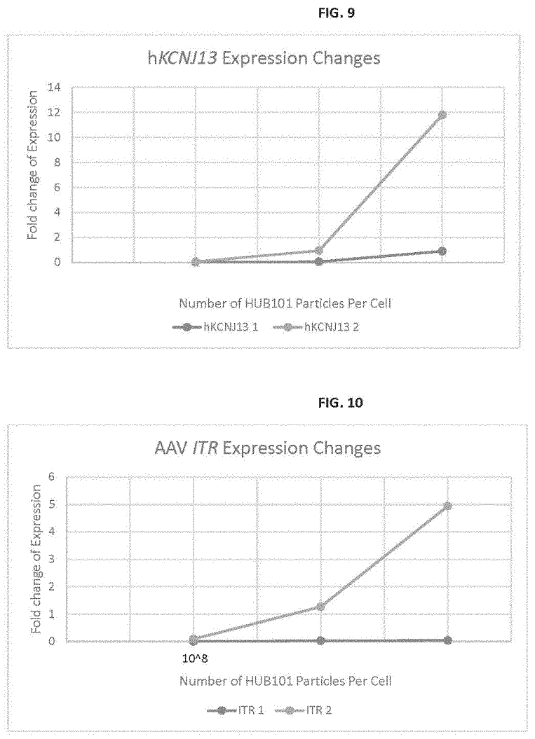

In one particularly suitable embodiment, the invention provides the construct as depicted in FIGS. 9 and 10 that comprises the AAV gene vector and is capable of being used to produce AAV virus particles for use in the present invention. One particular construct comprises or consists of SEQ ID NO:22 or a polynucleotide having at least 90% sequence identity to SEQ ID NO:22.

In another embodiment, the present disclosure provides a cell comprising the construct of described herein, wherein the cell is capable of producing AAV virus particles comprising the AAV vector capable of expressing Kir7.1 protein as described herein. Suitable cell types are known in the art and include, for example, mammalian cells. The cell may further comprise helper plasmids comprising AAV structural and functional proteins to produce AAV virus particles as known in the art.

In another embodiment, the present disclosure provides AAV virus particles made by the cells described herein. The AAV virus particles comprise the AAV vector and are capable of translocating the AAV vector into target cells, e.g., RPE cells, either in vitro or in vivo. As used herein, the term “virus particle” is used to refer to a virion consisting of nucleic acid surrounded by a protective coat of protein called a capsid. The term “viral vector” is commonly used to describe a virus particle used to deliver genetic material (e.g., the AAV vector and constructs of the present invention) into cells.

In one embodiment, the disclosure provides a construct comprising the AAV vector (SEQ ID NO:22) or a polynucleotide having at least 90% sequence similarity to SEQ ID NO:22 is provided. The construct comprising SEQ ID NO:22 is depicted in FIG. 9-10. The construct is preferably a plasmid. The construct depicted in FIGS. 9 and 10 comprises the 5′ITR (AAV 5′ inverted terminal repeat), VMD2 (RPE specific promoter), Kozak (Kozak translation initiation sequence which facilitates translation initiation of ATG start codon downstream of the Kozak sequence), Kir7.1 gene, WPRE (woodchuck hepatitis virus posttranscriptional regulatory element), BGH pA (bovine growth hormone polyadenylation signal), and 3′ITR (AV 3′ inverted terminal repeat). The plasmid further comprises additional polynucleotide sequences for its growth and propagation within bacterial cells, e.g., an antibiotic resistance (e.g., ampicillin) gene and an ori (plasmid origin of replication) to allow for plasmid replication and selection in E. Coli. The WPRE enhances virus stability in packaging cells, leading to high titer of packaged virus, and enhances higher expression of transgenes. The BGH pA signal allows transcription termination and polyadenylation of mRNA transcribed by Pol II RNA polymerase.

### Promoters

As used herein, the terms “promoter,” “promoter region,” or “promoter sequence” refer generally to transcriptional regulatory regions of a gene, which may be found at the 5′ or 3′ side of the polynucleotides described herein, or within the coding region of the polynucleotides, or within introns in the polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease 51), as well as protein binding domains (consensus sequences) responsible for the binding of RNA polymerase.

In some embodiments, the promoter is specific to the cell type in which Kir7.1 is to be expressed. For example, suitable cell types including retinal pigment epithelium, small intestinal cells, uterine cells, kidney cells, among others. The promoters may be specific to polarized cells, e.g., cells that have directionality and the Kir7.1 potassium pump plays a role in maintaining the polarization of the cells. Suitable promoters that may be used in a tissue specific manner include the RPE promoters (e.g., EF1a or VMD2) described and the promoters found below in Table 7. In some embodiments, the promoter is active in the retinal pigment epithelium (RPE) in the eye of a subject.

The “promoter” may be the endogenous promoter for the kcnj13 gene found, for example, in a subject. Alternatively, the promoter may be a heterologous promoter (i.e., a promoter for a non-kcnj13 gene). Heterologous promoters useful in the practice of the present invention include, without limitation, constitutive, inducible, temporally-regulated, developmentally regulated, chemically regulated, tissue-preferred and tissue-specific promoters.

Suitable heterologous promoters may include, without limitation, an EF1a promoter or a VMD2 promoter. An exemplary EF1a promoter is provided as SEQ ID NO:3. An exemplary VMD2 promoter is provided as SEQ ID NO:4 and SEQ ID NO:24. Suitable EF1a promoters may also include variants of the EF1a promoter provided as SEQ ID NO:3 having at least 60%, 65%, 70%, 75%, 80%, 85%, 90%, 91%, 92%, 93%, 94%, 95%, 96%, 97%, 98%, 99%, or 100% sequence identity to SEQ ID NO:3. Suitable VMD2 promoters may also include variants of the VMD2 promoter provided as SEQ ID NO:4 or 24 having at least 60%, 65%, 70%, 75%, 80%, 85%, 90%, 91%, 92%, 93%, 94%, 95%, 96%, 97%, 98%, 99%, or 100% sequence identity to SEQ ID NO:4 or 24.

Regarding polynucleotides such the promoters and Kir7.1 polynucleotides described herein, the phrases “% sequence identity,” “percent identity,” or “% identity” refer to the percentage of base matches between at least two polynucleotide sequences aligned using a standardized algorithm. Methods of polynucleotide sequence alignment are well-known. A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn.”

In some embodiments, the disclosed polynucleotides encoding a Kir7.1 polypeptide are operably connected to the promoter. As used herein, a polynucleotide is “operably connected” or “operably linked” when it is placed into a functional relationship with a second polynucleotide sequence. For instance, a promoter is operably linked to a polynucleotide if the promoter is connected to the polynucleotide such that it may effect transcription of the polynucleotides. In various embodiments, the polynucleotides may be operably linked to at least 1, at least 2, at least 3, at least 4, at least 5, or at least 10 promoters.

As used herein, a “Kir7.1 polypeptide” or “KCNJ13 polypeptide” are used interchangeably and refer to an inward rectifier potassium channel characterized by a greater tendency to allow potassium to flow into the cell rather than out of it. A human Kir7.1 polypeptide is provided as SEQ ID NO:1. A Kir7.1 polypeptide may also be a variant or homolog of the human Kir7.1 polypeptide provided as SEQ ID NO:1 having at least 60%, 65%, 70%, 75%, 80%, 85%, 90%, 91%, 92%, 93%, 94%, 95%, 96%, 97%, 98%, 99%, or 100% sequence identity to SEQ ID NO:1.

Regarding Kir7.1 polypeptides, the phrases “% sequence identity,” “percent identity,” or “% identity” refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The Kir7.1 polypeptides disclosed herein may include “variant” polypeptides, “mutants,” and “derivatives thereof” As used herein the term “wild-type” is a term of the art understood by skilled persons and means the typical form of a polypeptide as it occurs in nature as distinguished from variant or mutant forms. As used herein, a “variant, “mutant,” or “derivative” refers to a polypeptide molecule having an amino acid sequence that differs from a reference protein or polypeptide molecule. A variant or mutant may have one or more insertions, deletions, or substitutions of an amino acid residue relative to a reference molecule. For example, a Kir7.1 polypeptide mutant or variant may have one or more insertions, deletions, or substitution of at least one amino acid residue relative to the Kir7.1 “wild-type” polypeptides disclosed herein. The polypeptide sequence of a “wild-type” Kir7.1 polypeptides is provided as SEQ ID NO:1. This sequence may be used as a reference sequence.

The Kir7.1 polypeptides provided herein may be full-length polypeptides or may be fragments of the full-length polypeptide. As used herein, a “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 350 contiguous amino acid residues of a reference polypeptide, respectively. In some embodiments, a fragment may comprise at least 5, 10, 15, 20, 25, 30, 40, 50, 60, 70, 80, 90, 100, 150, or 250 contiguous amino acid residues of a reference polypeptide. Fragments may be preferentially selected from certain regions of a molecule. The term “at least a fragment” encompasses the full length polypeptide. A fragment of a Kir7.1 polypeptide may comprise or consist essentially of a contiguous portion of an amino acid sequence of a full-length Kir7.1 polypeptide (See SEQ ID NO:1). A fragment may include an N-terminal truncation, a C-terminal truncation, or both truncations relative to the full-length Kir7.1 polypeptide.

A “deletion” in a Kir7.1 polypeptide refers to a change in the amino acid sequence resulting in the absence of one or more amino acid residues. A deletion may remove at least 1, 2, 3, 4, 5, 10, 20, 50, 100, 200, or more amino acids residues. A deletion may include an internal deletion and/or a terminal deletion (e.g., an N-terminal truncation, a C-terminal truncation or both of a reference polypeptide).

“Insertions” and “additions” in a Kir7.1 polypeptide refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, 200, or more amino acid residues. A variant of a Kir7.1 polypeptide may have N-terminal insertions, C-terminal insertions, internal insertions, or any combination of N-terminal insertions, C-terminal insertions, and internal insertions.

The amino acid sequences of the Kir7.1 polypeptide variants, mutants, derivatives, or fragments as contemplated herein may include conservative amino acid substitutions relative to a reference amino acid sequence. For example, a variant, mutant, derivative, or fragment polypeptide may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

The disclosed variant and fragment Kir7.1 polypeptides described herein may have one or more functional or biological activities exhibited by a reference polypeptide (e.g., one or more functional or biological activities exhibited by a wild-type Kir7.1 polypeptide (i.e, SEQ ID NO:1). Suitably, the disclosed variant or fragment Kir7.1 polypeptide retains at least 20%, 40%, 60%, 80%, or 100% of the potassium conductance properties of the reference polypeptide. As used herein, a “functional fragment” of a Kir7.1 polypeptide is a fragment of, for example, the polypeptide of SEQ ID NO:1 that retains at least 20%, 40%, 60%, 80%, or 100% of the potassium conductance properties of the full-length ADH polypeptide.

Furthermore, it will be readily apparent to a person of ordinary skill in the art that additional Kir7.1 polypeptide variants may be created by aligning Kir7.1 polypeptide sequences from two or more species. Based on these alignments, a person of ordinary skill in the art may identify various amino acid residues that may be altered (i.e. substituted, deleted, etc.) without substantially affecting the potassium conductance properties of the polypeptide. For example, a person of ordinary skill in the art would appreciate that substitutions in a reference Kir7.1 polypeptide could be based on alternative amino acid residues that occur at the corresponding position in other Kir7.1 polypeptides from other species.

### Therapeutic Compositions

In another aspect, the present invention relates to therapeutic compositions. The therapeutic compositions may include any of the gene therapy vectors described herein and a pharmaceutically-acceptable carrier. The therapeutic compositions may include a pharmaceutically-acceptable carrier, excipient, or diluent, which are nontoxic to the cell or subject being exposed thereto at the dosages and concentrations employed. Often a pharmaceutical diluent is in an aqueous pH buffered solution. Examples of pharmaceutically-acceptable carriers or excipients may include, without limitation, water, buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ brand surfactant, polyethylene glycol (PEG), and PLURONICS™ surfactant.

### Methods of Treatment

In a further aspect of the present invention, methods of treating a subject having a condition associated with insufficient expression or function of a Kir7.1 polypeptide are provided. The methods may include administering a therapeutically effective amount of any one of the gene therapy vectors described herein or any one of the therapeutic compositions described herein to the subject. As used herein, the terms “subject” and “patient” are used interchangeably to refer to both human and nonhuman animals. The term “nonhuman animals” of the disclosure may include mammals and non-mammals, such as nonhuman primates, sheep, dog, cat, horse, cow, pig, mice, rats, and the like. In some embodiments, the subject is a human patient. The subject may be a human patient having cells (i.e., RPE cells) that exhibit insufficient expression or function of a Kir7.1 polypeptide.

Conditions associated with insufficient expression or function of a Kir7.1 polypeptide may include conditions in which a subject has reduced or eliminated Kir7.1 expression or function in or outside a cell as compared to a control. As used herein, a “control” may include subjects having wildtype Kir7.1 function. For example, in some embodiments, a control may be a subject having a wildtype kcnjl3 gene that does not include any loss-of-function mutations in either the non-coding regulatory sequences (i.e., promoter, enhancers, etc.) controlling the expression of the KCNJ13 gene or in the coding region of the kcnj13 gene (i.e., SEQ ID NOS: 1 and 2).

Subjects may have several “cell” types that may display insufficient expression or function of a Kir7.1 polypeptide. As used herein, a “cell” may refer to cells that normally express a Kir7.1 polypeptide in a wild-type subject. Suitable cells may include, without limitation, eye cells such as retinal cells or retinal pigment epithelium (RPE) cells. Kir7.1 is also expressed in epithelial cells of various organs including kidney, thyroid, CNS neurons, ependymal cells, choroid plexus epithelium, spinal cord, myometrial smooth muscle, small intestine, neural regions of the gastric mucosa as well as gastric parietal cells, and also in the lung, prostate, liver, pancreas, cochlear nucleus, testis and ovaries.

In some embodiments, the condition associated with insufficient expression or function of a Kir7.1 polypeptide may be associated with at least one loss-of-function mutation in a KCNJ13 gene. The human KCNJ13 gene is provided as UniProt 060928. The KCNJ13 gene in other non-human subjects may be identified by using homology searching methods well known in the art. Suitable loss-of-function mutations in the KCNJ13 gene may include at least one substitution to the Kir7.1 protein provided as SEQ ID NO:1 selected from the group consisting of W53Ter, Q116R, 1120T, T1531, R162Q, R166Ter, L241P, E276A, 51051, and G219Ter. In some embodiments, the condition associated with insufficient expression or function of a Kir7.1 polypeptide may be, without limitation, Leber Congenital Amaurosis 16 (LCA16), retinitis pigmentosa, or Snowflake Vitreoretinal Degeneration (SVD). In some embodiments, the vectors described herein can increase expression of KCNJ13 in RPE cells, reducing RPE cell loss and cell function. In another embodiment, the vectors described herein can be used in the treatment of age-related macular degeneration.

Thus, in some embodiments, the present disclosure provides methods of expressing a KCNJ13 gene in a retinal pigment epithelium (RPE) cell. The RPE may be in vivo in a subject. The vector may be administered intraocularly, for example, subretinally or intravitreally. In some embodiments, the vector may be targeted to the macula. In another embodiment, the RPE may be an autologous induced pluripotent stem cell (iPSC)-derived RPE in vitro. The iPSC-derived RPE comprising the vector can be used for transplantation into a subject in need thereof. In one embodiment, the subject may be a subject having age-related macular degeneration. Methods of differentiating iPSC cells into RPE are known in the art. The vector or RPE cell comprising the vector may be used to reduce or halt progression of age-related macular degeneration. Not to be bound by any theory, but increase expression of KCNJ13 protein in the apical membrane of RPE may help to decrease the risk of retinal detachment and/or reduce or halt the progression of age-related macular degeneration.

Methods of transplanting RPE cells into a subject are known and practiced by one skilled in the art.

“Treating” the condition associated with insufficient expression or function of a Kir7.1 polypeptide includes, without limitation, increasing the levels of functional Kir7.1 polypeptide in or outside a cell in a subject. It would be understood by one skilled in the art that an increase in the amount of functional Kir7.1 may only need to be an increase of at least about 10%, preferably at least about 20%, alternatively about 30%, which may result in the proper functioning of the potassium channel within the cell in which it is expressed, leading to alleviation of one or more symptoms of the disease. For example, the ratio of functional to non-functional Kir7.1 within the cell needs to be sufficient to allow for proper functioning of the potassium channel, and may vary depending of cell type and location.

A “therapeutically effective amount” or an “effective amount” as used herein means the amount of a composition that, when administered to a subject for treating a state, disorder or condition is sufficient to effect a treatment (as defined above). The therapeutically effective amount will vary depending on the compound, formulation or composition, the disease and its severity and the age, weight, physical condition and responsiveness of the subject to be treated.

The compositions (i.e. gene therapy vectors and/or therapeutic compositions) described herein may be administered by any means known to those skilled in the art, including, without limitation, locally or systemically, including, for example, intraocularly, intravitreal topically, intranasally, intramuscularly, or subcutaneously. When administered intraocularly, in some embodiments, the compositions (i.e. gene therapy vectors and/or therapeutic compositions) may be administered subretinally by, for example, injection to at least one retina of the subject. In the retina, the targeted region for delivery of the compositions (i.e. gene therapy vectors and/or therapeutic compositions) may include the central superior retina or macula.

Such intraocular administration routes are within the skill in the art; see, e.g., and Acheampong A A et al, 2002, supra; and Bennett et al. (1996), Hum. Gene Ther. 7: 1763-1769 and Ambati J et al., 2002, Progress in Retinal and Eye Res. 21: 145-151, the entire disclosures of which are herein incorporated by reference. The general method for intravitreal injection may be illustrated by the following brief outline. This example is merely meant to illustrate certain features of the method, and is in no way meant to be limiting. Procedures for intravitreal injection are known in the art (see, e.g., Peyman, G. A., et al. (2009) Retina 29(7):875-912 and Fagan, X. J. and Al-Qureshi, S. (2013) Clin. Experiment. Ophthalmol. 41(5):500-7).

Briefly, a subject for intravitreal injection may be prepared for the procedure by pupillary dilation, sterilization of the eye, and administration of anesthetic. Any suitable mydriatic agent known in the art may be used for pupillary dilation. Adequate pupillary dilation may be confirmed before treatment. Sterilization may be achieved by applying a sterilizing eye treatment, e.g., an iodide-containing solution such as Povidone-Iodine (BETADINETM). A similar solution may also be used to clean the eyelid, eyelashes, and any other nearby tissues (e.g., skin). Any suitable anesthetic may be used, such as lidocaine or proparacaine, at any suitable concentration. Anesthetic may be administered by any method known in the art, including without limitation topical drops, gels or jellies, and subconjuctival application of anesthetic.

Prior to injection, a sterilized eyelid speculum may be used to clear the eyelashes from the area. The site of the injection may be marked with a syringe. The site of the injection may be chosen based on the lens of the patient. For example, the injection site may be 3-3.5 mm from the limus in pseudophakic or aphakic patients, and 3.5-4 mm from the limbus in phakic patients. The patient may look in a direction opposite the injection site.

In another embodiment, the vector is provided subretinally. It will be appreciated that the specific dosage administered in any given case will be adjusted in accordance with the composition or compositions being administered, the disease to be treated or inhibited, the condition of the subject, and other relevant medical factors that may modify the activity of the compositions or the response of the subject, as is well known by those skilled in the art. For example, the specific dose for a particular subject depends on age, body weight, general state of health, diet, the timing and mode of administration, medicaments used in combination and the severity of the particular disorder to which the therapy is applied. Dosages for a given patient can be determined using conventional considerations, e.g., by customary comparison of the differential activities of the compositions described herein and of a known agent, such as by means of an appropriate conventional pharmacological or prophylactic protocol. The maximal dosage for a subject is the highest dosage that does not cause undesirable or intolerable side effects. The number of variables in regard to an individual treatment regimen is large, and a considerable range of doses is expected. The route of administration will also impact the dosage requirements.

The effective dosage amounts described herein refer to total amounts administered, that is, if more than one composition is administered, the effective dosage amounts correspond to the total amount administered. The compositions can be administered as a single dose or as divided doses. For example, the composition may be administered two or more times separated by 4 hours, 6 hours, 8 hours, 12 hours, a day, two days, three days, four days, one week, two weeks, or by three or more weeks.

The compositions (i.e. gene therapy vectors and/or therapeutic compositions) described herein may be administered one or more times to the subject to effectively increase the levels of functional Kir7.1 polypeptide in or outside a cell in a subject. The compositions (gene therapy vectors or therapeutic compositions) may be administered based on the number of copies of the expression construct encoding a Kir7 polypeptide delivered to the subject. The subject may be administered between 10⁶ and 10¹⁴, or between 10⁸ and 10¹², or between 10⁹ and 10¹¹, or any range therein copies. In embodiments where the gene therapy vector is a viral vector, the subject may be administered between 10⁶ and 10¹⁴, or between 10⁸ and 10¹², or between 10⁹ and 10¹¹, or any range therein viral genomes.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference in their entirety, unless explicitly indicated otherwise. The present disclosure shall control in the event there are any disparities between any definitions and/or description found in the cited references.

Unless otherwise specified or indicated by context, the terms “a”, “an”, and “the” mean “one or more.” For example, “a protein” or “an RNA” should be interpreted to mean “one or more proteins” or “one or more RNAs,” respectively.

The following examples are meant only to be illustrative and are not meant as limitations on the scope of the invention or of the appended claims.

### EXAMPLES

### Example 1

### Kir7.10 Gene Therapy In Cell and In Vivo

Leber Congenital Amaurosis (LCA) is an inherited pediatric blindness that is associated with at least 21 different genes. In our previous work, we showed that the molecular mechanisms underlying LCA16 is due to a nonsense mutation in the KCNJ13 gene resulting in a nonfunctional Kir7.1 ion channel. Using either read-through or gene augmentation, we demonstrated rescued Kir7.1 channel function in patient-derived iPSC-RPE cells via a precision medicine approach.

Mutations in at least 21 genes that are expressed in the outer retinal photoreceptors and retinal pigment epithelium (RPE) cause a form of inherited blindness known as Leber

Congenital Amaurosis (LCA), from birth and early childhood. Within the last decade, autosomal recessive mutations in the KCNJ13 gene (603203 on chromosome locus 2q37.1) have been identified in patients with an LCA phenotype (LCA16 OMIM-614186, the 16^{th} gene shown to cause LCA)¹⁻³. LCA16 pathogenic allelic variants include c.158G>A (p.Trp53Ter), c.359T>C (p.Iso120Thr), c.458C>T (p.Thr153Iso), c.496C>T (p.Arg166Ter), and c.722T>C (p.Leu241Pro)¹′²′⁴. In addition, the compound heterozygous KCNJ13 mutations c.314 G>T (p.Ser105Iso) and c.655C>T (p.G219Ter) are known to cause early-onset retinal dystrophy in an LCA patient⁵. An autosomal dominant KCNJ13 mutation, c.484C>T (p.Arg162Trp), causes early-onset blindness called snowflake vitreoretinal degeneration (SVD OMIM-193230)⁶.

In the retina, Kir7.1 is expressed exclusively in cell apical processes of RPE, in which it modulates retinal function and health.

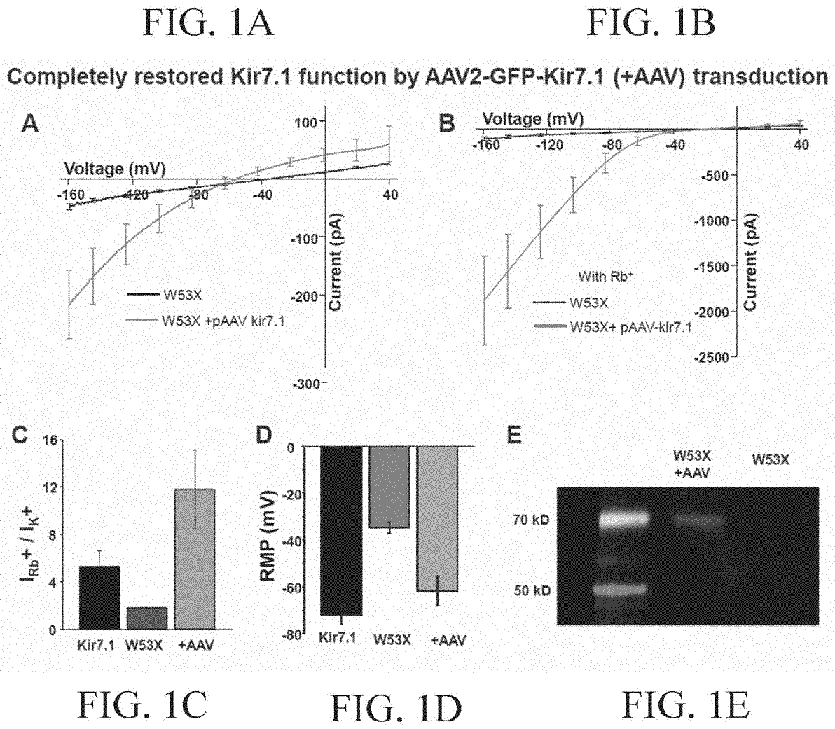

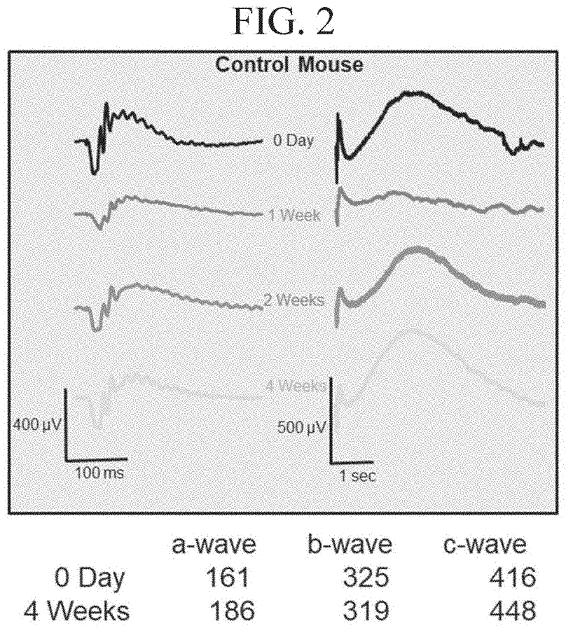

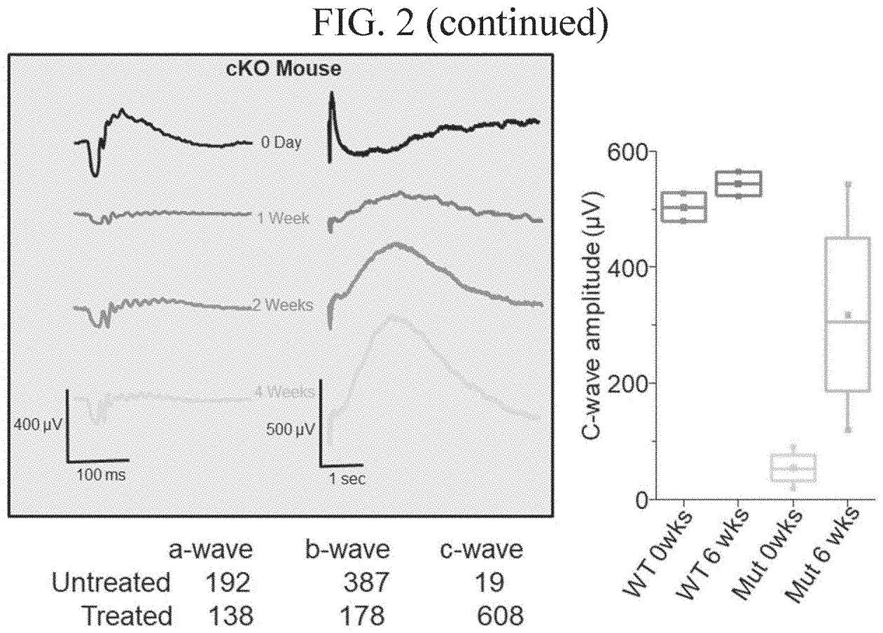

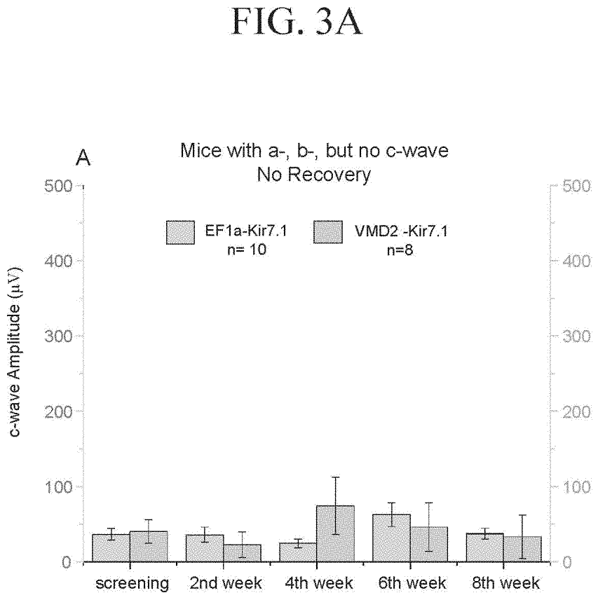

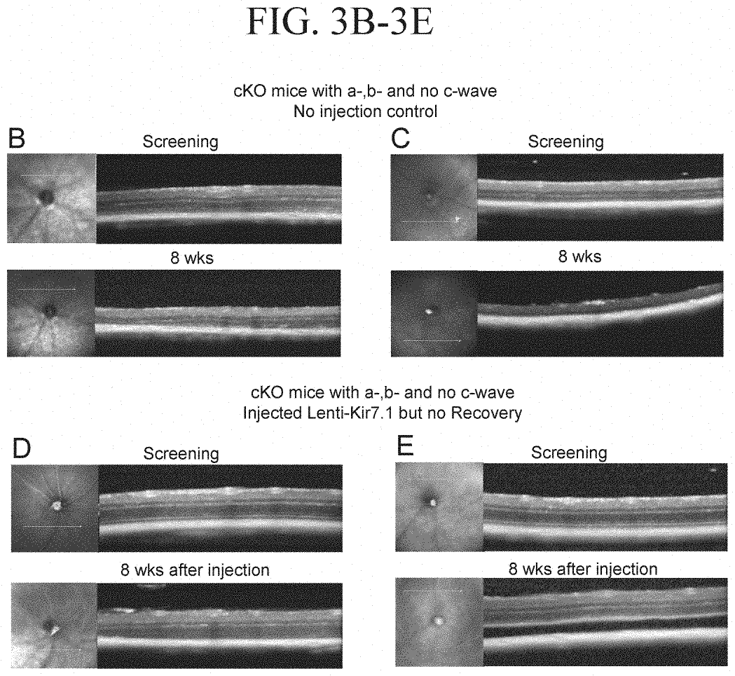

Our previous results demonstrated the efficacy of gene therapy in a cell culture model of LCA16 by testing the ability of AAV-Kir7.1 to rescue the physiological defects in CHO cells harboring a W53X mutation in the KCNJ13 gene. FIG. 1 shows gene augmentation of W53X mutant expressing CHO cells had recovery of average inwardly rectifying K+ current (FIG. 1A. IV plot in red trace) compared to no current before (FIG. 1A. plot in black trace). (FIG. 1B) Average higher Rb+current (red trace) in W53X mutant expressing cells after gene augmentation. (FIG. 1C) Net increase in Rb+permeability increased (Blue) through Kir7.1 channel after gene augmentation. (FIG. 1D) Complete recovery of resting membrane potential (RMP) after AAV-Kir7.1 transduction of W53X expressing cells represented as blue box. (FIG. 1E) Western blot results showing expression of full length protein product after gene augmentation in lane W53X +AAV (red band).To test the efficacy of gene therapy in vivo, both wild-type and a mouse lacking the KCNJ13 gene were tested. In FIG. 2 left box is an example of a wild type mouse that received 2 μl of Lenti-EF1a-eGFPKir7.1 by sub-retinal injection. Electrophysiological results are obtained before (black trace) and 1 (blue trace), 2 (red trace), and 4 (green trace) weeks post injection. In FIG. 2 left box, retina responses recorded as normal a- and b-wave are shown on the left and RPE cell response c-wave is shown on the right. Only in the 1^{st} week after injection there was a reduction in retina response otherwise there was hardly any effect of gene therapy on electrophysiological outcome. In FIG. 2 right box we show results from mice lacking KCNJ13 l gene that received 2 μl of Lenti-EF1a-eGFPKir7.1. On the right panel is the RPE response of c-wave, that was completely abolished in these mice (black trace) with slight reduction in a- and b-wave shown in the left panel. Immediately post gene-therapy, we noticed increase in c-wave response starting a week after injection (blue trace on the right panel). Traces show continued increase in c-wave during the following 2 (red trace) and 4 (green) weeks post gene therapy. Average measurements in 4 wild-type and four mice lacking KCNJ13 gene is shown as box plot with significant recovery in c-wave and no effect on wild-type mice vision. Numbers below the figure shows actual amplitude of a-, b- and c-wave measurements in wild-type and mice lacking Kcnj13.

### Material and Methods

### Animals

To elucidate the physiological role of KCNJ13 gene in the RPE cells, in vivo, we used a strain that is lacking this gene. Vision in these mice was measured using electroretinography (ERG). The mice were housed and bred at the University of Wisconsin Biotron (Madison, Wis.)

### Electroretinography

The mice were dark adapted overnight prior to performing ERG. The mice were anesthetized with Ketamine/Xylazine (80:16 mg/kg) cocktail injected intra-peritoneally. While maintaining the body temperature at 37° C. with a heating pad, the pupil of the mouse was dilated with a drop of tropicamide (Bausch+Lomb, Rochester, N.Y.). ERGs were performed using the Espion recording system (Diagnosys) by placing a corneal contact lens (Ocusciences Inc., MO) on the dilated eyes along with Gonak, a 2.5% hypromellose ophthalmic demulcent solution (GONIOVISC, HUB Pharmaceuticals, LLC, CA). A reference and the ground electrode were placed in the mouth and the back respectively. The protocol for ERG consisted of recordings from flash intensities from 0.1 to 30 cd.s.m-2 and 60Hz line noise was removed using the filter. For c-wave measurements, we used a 5 msec flash of 25 cd.s.m-2 intensity to acquire data during a 5 sec interval. ERG analysis was performed on the mice before and after the sub-retinal injection.

### Sub-Retinal Injection

The KCNJ13 knockout mice with no c-waveforms were used for this purpose. The mice were maintained under tightly controlled temperature (23 ±5° C.), humidity (40-50%) and light/dark (12/12 h) cycle conditions in 200 lux light environment. Prior to the injection, the mice were anesthetized, and pupils were dilated as described above. 2 μl of Lentivirus or Adeno-associated virus (AAV) carrying the functional full length KCNJ13 gene fused with eGFP and driven by EF1a or VMD2 promoters were delivered to the RPE cells through sub-retinal injection using a 10mm 34 gauge needle. We used a 10 μl NanoFil syringe and UMP3, NanoFil RPE-KIT and Micro4 controller (World precision Instruments, Inc., Sarasota, FL). ERG was performed on these mice at 1 wk, 2 wks, 4 wks and 8 wks post injection and data were analyzed.

### Transgene Expression Detection

eGFP fluorescence was detected using confocal microscopy after preparing a flat mount of the isolated RPE. Eyes from the Lentivirus/AAV carrying eGFP-KCNJ13 gene injected mice were retrieved one-week post injection. Enucleated eyes from the sacrificed mice were rinsed twice with PBS, a puncture was made at ora serrata with a 28-gauge needle and the eyes were opened along the corneal incisions. The lens was then carefully removed. The eye cup was flattened making incisions radially to the center resulting in a “starfish” appearance. The retina was then separated gently from the RPE layer. The separated RPE and retina were flat mounted on the cover-glass slide and were imaged with NIS-Elements using a Nikon C2 confocal microscope (Nikon Instruments Inc., Mellville, N.Y.). We used 488 nm Diode Lasers for green excitation and images were captured by Low Noise PMT C2 detectors in a Plan Apo VC 20X/0.75, 1 mm WD lens.

Differentiation of hiPSC-RPE. Fibroblasts from two subjects were reprogrammed to induced pluripotent stem cells and cultured using established methods¹⁻³. One of the subjects was an LCA16 patient with two copies of the Trp53Ter autosomal recessive mutation in the KCNJ13 gene, and the second subject was heterozygous for this mutation. The hiPSC lines were differentiated to RPE using protocols described earlier²⁻⁵. Briefly, hiPSCs were cultured either on mouse embryonic fibroblasts (MEFs) in iPS cell media (Dulbecco's modified Eagle's medium (DMEM): F12 (1:1), 20% Knockout Serum, 1% minimal essential medium (MEM) non-essential amino acids, 1% GlutaMAX, β-mercaptoethanol, 20 ng/ml FGF-2), or on Matrigel® with mTeSR1 media. Cells were lifted enzymatically and grown as embryoid bodies (EBs) in iPS medium without FGF-2, and at day 4, changed to neural induction medium (NIM; DMEM: F12; 1% N2 supplement, 1% MEM non-essential amino acids, 1% L-Glutamine, 2 μg/m1 Heparin), or in mTeSR1 and gradually transitioned to NIM by day 4. There were no differences observed in RPE differentiation between these two approaches. At day 7, free-floating Ebs were plated on laminin-coated culture plates to continue differentiation as adherent culture. At day 16, the 3D neural structures were removed, and medium was switched to retinal differentiation medium (DMEM/F12 (3:1), 2% B27 supplement (without retinoic acid), 1% Antibiotic-Antimycotic). Remaining adhered cells were allowed to continue differentiation for an additional 45 days, followed by microdissection and passaging of pigmented RPE patches to obtain purified monolayers of RPE as described earlier⁵. MEFs, Matrigel® and FGF-2 were purchased from WiCell (Madison, Wis.), and all other tissue culture reagents were purchased from ThermoFisher.

RT-PCR and Restriction Fragment Length Polymorphism (RFLP). Total RNA was isolated from the mature hiPSC-RPE cells from both patient and the carrier using the Rneasy® kit according to manufacturer's instructions (Qiagen). The quality and the concentration of the isolated RNA was measured using a Nanodrop (ThermoFisher) and 200 ng of RNA was used for cDNA synthesis using the Superscript III first strand cDNA synthesis kit according to manufacturer's instructions (ThermoFisher). PCR was performed with MyTaqHS master mix (Bioline) in a final volume of 25 μl with the following conditions: 95° C. for 5 min followed by 35 cycles of denaturation at 95° C. for 15 sec, annealing at 55° C. for 30 sec, and extension at 72° C. for 30 sec. A final extension step was done for 10 min at 72° C. and amplification products were visualized by electrophoresis on a 2% agarose gel containing Midori green advanced stain (Nippon Genetics Europe). For RFLP assay PCR was performed as described with primers specific to the full length KCNJ13 mRNA (Fwd 5′-GCTTCGAATTCCGACAGCAGTAATTG-3′ (SEQ ID NO:7) and Rev 5′-ATCCGGTGGATCCTTATTCTGTCAGT-3′ (SEQ ID NO:8). The PCR products were then digested by NheI restriction enzyme (ThermoFisher) and visualized by electrophoresis on a 2% agarose gel containing Midori green advanced stain (Nippon Genetics Europe).

Transmission Electron Microscopy. Monolayers of hiPSC-RPE on transwell inserts (Corning, Cat#3470) were fixed in a solution of 2.5% glutaraldehyde, 2.0% paraformaldehyde in 0.1 M sodium phosphate buffer (PB), pH 7.4 for ˜1 hr at room temperature (RT). Samples were rinsed 5×5 minutes in 0.1 M PB. The rinsed cultures were then post-fixed in 1% Osmium Tetroxide (0504), 1% potassium ferrocyanide in PB for 1 hr at RT. Following post-fixation, samples were rinsed in PB, as before, followed by 3×5 minute rinses in distilled water to clear the phosphates. The samples were then stained en bloc in uranyl acetate for 2 hrs at RT and dehydrated using ethanol series. The membrane was cut from the transwell support, placed in an aluminum weighing dish, transitioned in propylene oxide (PO) and allowed to polymerize in fresh PilyBed 812 (Polysciences Inc. Warrington, Pa.). Ultrathin sections were prepared from these polymerized samples and processed before capturing and documenting the images with FEI CM120 transmission electron microscope mounted with AMT BioSprint12 (Advanced Microscopy Techniques, Corp. Woburn, Mass.) digital camera.

### Example 2

### AAV Viral Vector Construction

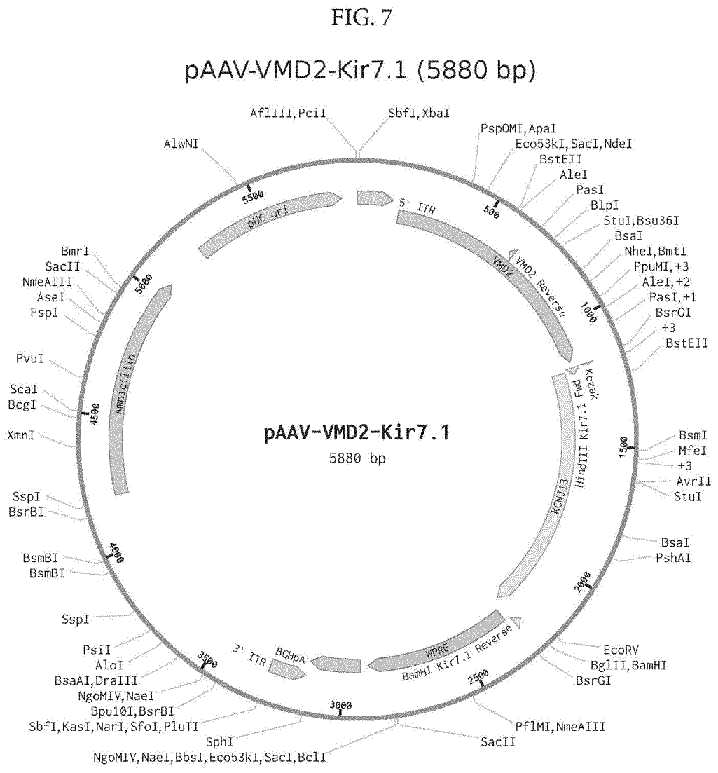

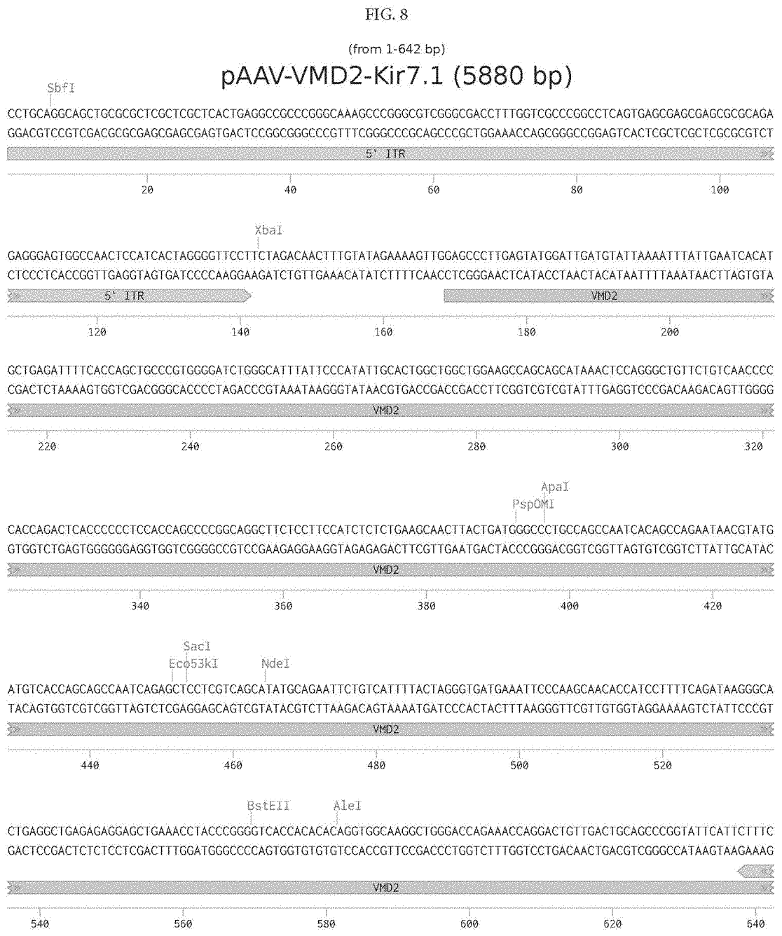

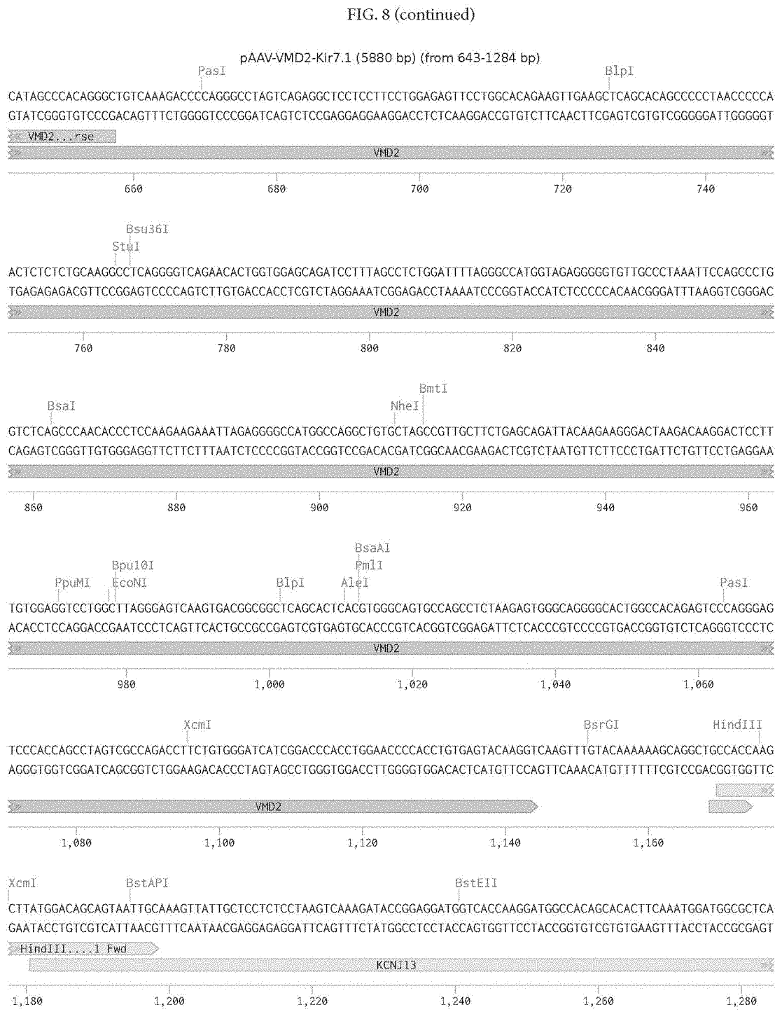

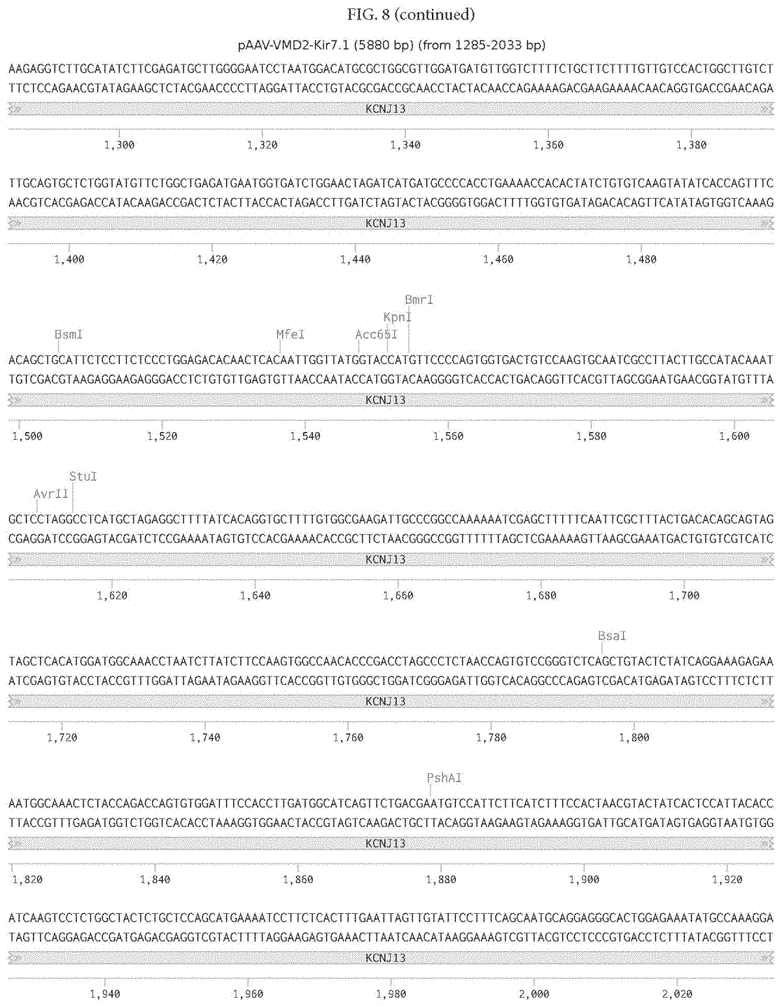

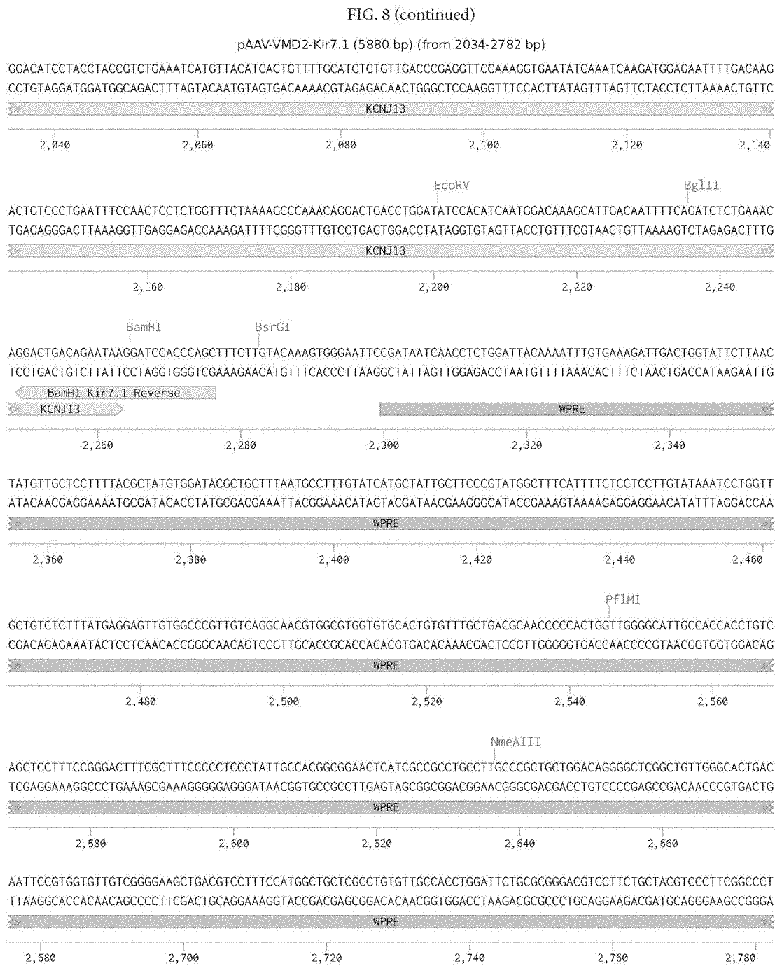

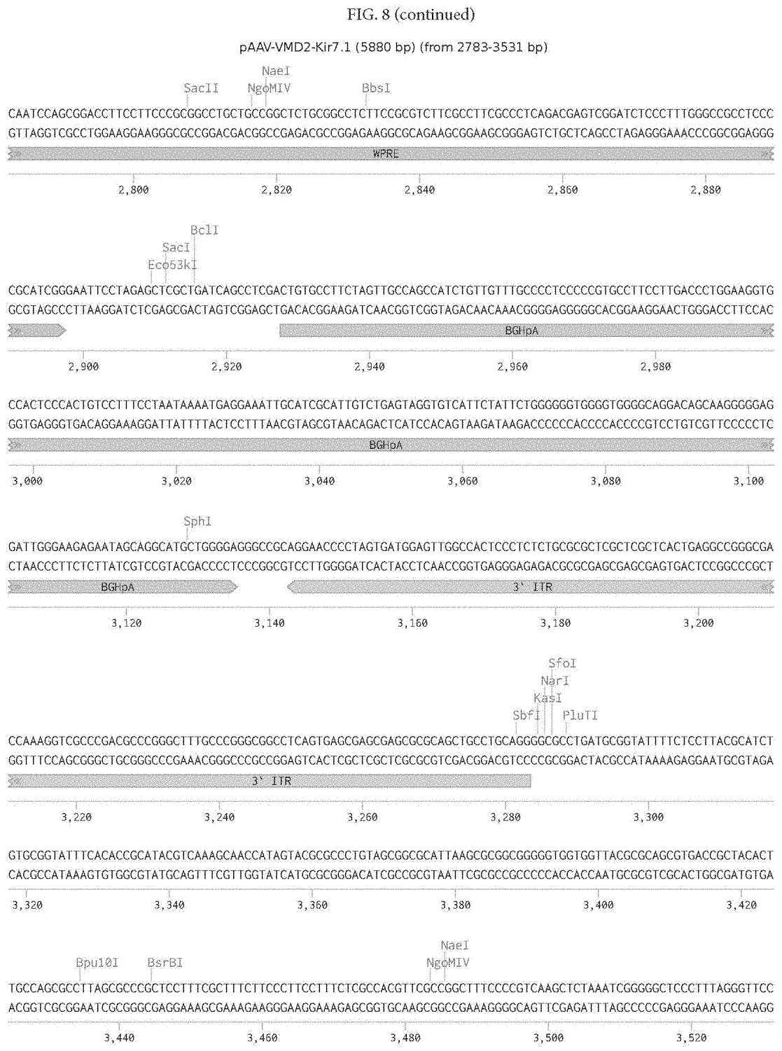

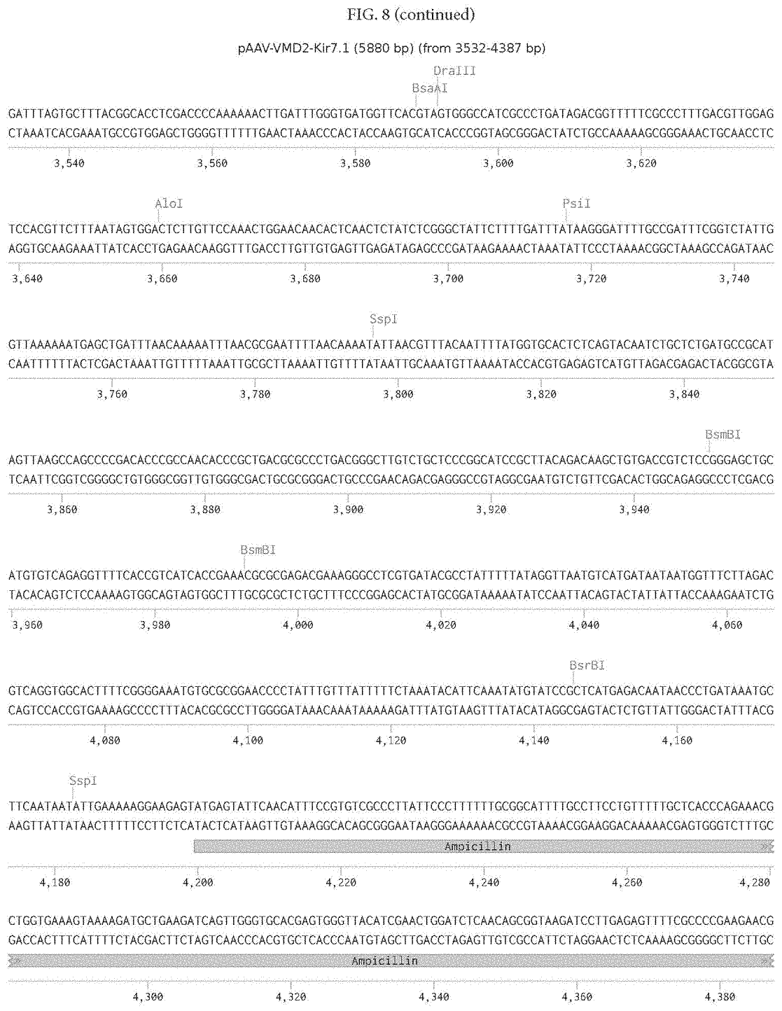

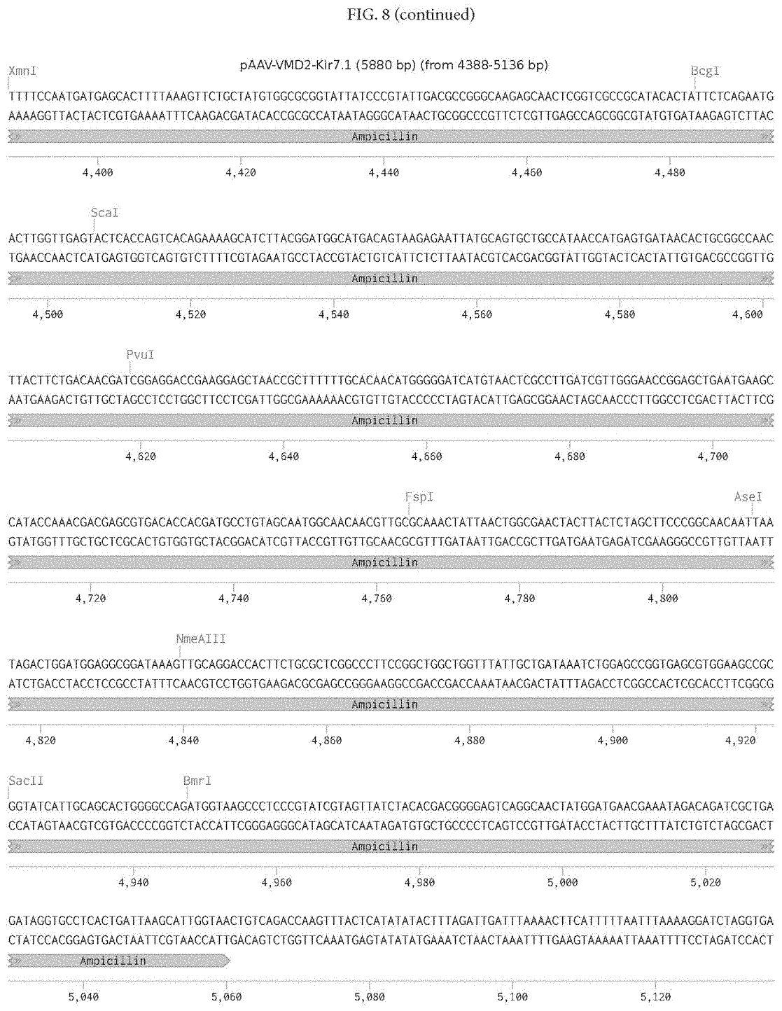

AAV vector for delivery of Kir7.1 protein is depicted in FIG. 7 (vector map) and the sequence was verified as depicted in FIG. 8.

### AAV Viral Vector Packaging

The adeno-associated virus (AAV) vector system is a popular and versatile tool for in vitro and in vivo gene delivery. AAV is effective in transducing many mammalian cell types, and, unlike adenovirus, has very low immunogenicity, being almost entirely nonpathogenic in vivo. This makes AAV the ideal viral vector system for many animal studies.

An AAV vector is first constructed as a plasmid in E. coli. It is then transfected into packaging cells along with helper plasmids, where the region of the vector between the two inverted terminal repeats (ITRs) is packaged into live virus. When the virus is added to target cells, the double-stranded linear DNA genome is delivered into cells where it enters the nucleus and remains as episomal DNA without integration into the host genome. Any gene(s) placed in-between the two ITRs are introduced into target cells along with the rest of viral genome.

A major practical advantage of AAV is that in most cases AAV can be handled in biosafety level 1 (BSL1) facilities. This is due to AAV being inherently replication-deficient, producing little or no inflammation, and causing no known human disease.

We found that the AAV2 serotype may be used to effectively transduce retinal pigment epithelium (RPE) cells either in vitro or in vivo.

### Example 3

### AAV27M8 Transduces Both iPSC-RPE and Mouse RPE Cells In Vivo Following Intravitreal Delivery

Prior work has shown iPSC-RPE transduced by lentiviruses but not AAV serotypes resulted in gene delivery to RPE cells. This Example demonstrates successful targeting and expression by an AAV vector (AAV27M8) in both iPSC-RPE cell and in vivo.

Construction of an AAV27M8 vector encoding Kir7.1 was made (SEQ ID NO:21 and SEQ ID NO:22, SEQ ID NO:21 comprising EGFP for easy detection). As demonstrated in FIG. 4, the inventors have successfully expressed KCNJ13 gene (as shown for GFP expression) in mature human iPSC-RPE cells in culture.

AAV27M8, as in SEQ ID NO:21 comprises EGFP for the ability to track when injected. The description of the different regions of SEQ ID NO:21 can be found in Table 1:

TABLE 1
description of AAV27M8 with EGFP

| | | Size | | | |
|---|---|---|---|---|---|
| Feature | Position | (bp) | Type | Description | Application Notes |
| 5′ITR | 1-141 | 141 | ITR | AAV 5′ inverted | Allows replication of viral |
| | | | | terminal repeat | genome and its packaging |
| | | | | (function equivalent | into virus. |
| | | | | of wild-type 5′ ITR) | |
| {VMD2} | 169-1144 | 976 | Promoter | None | None |
| Kozak | 1169-1174 | 6 | Misc. | Kozak translation | Facilitates translation |
| | | | | initiation sequence | initiation of ATG start codon |
| | | | | | downstream of the Kozak |
| | | | | | sequence. |
| {EGFP/HindhKCNJ13[NM_002242.4]} | 1175-2983 | 1809 | ORF | None | None |
| WPRE | 3014-3611 | 598 | Regulatory | Woodchuck | Enhances virus stability in |
| | | | Element | hepatitis virus | packaging cells, leading to |
| | | | | posttranscriptional | high titer of packaged virus, |
| | | | | regulatory element | enahcnes higher expression |
| | | | | | of transgenes. |
| BGH pA | 3642-3849 | 208 | PolyA_signal | Bovine growth | Alls transcription termination |
| | | | | hormone | and polyadenylation of |
| | | | | polyadenylation | mRNA transcribed by |
| | | | | signal | Pol II RNA polymerase. |
| 3′ ITR | compliment | 141 | ITR | AAV 3′ inverted | Allows replication of viral |
| | (3857-3997) | | | terminal repeat | genome and its packaging |
| | | | | | into virus. |
| Ampicillin | 4914-5774 | 861 | ORF | Ampicillin | Allows E. coli to be resistant |
| | | | | resistance gene | to ampicillin. |
| pUC ori | 5945-6533 | 589 | Rep_origin | pUC origin of | Facilitates plasmid replication |
| | | | | replication | in E coli; regulates high-copy |
| | | | | | plasmid number (500-700). |

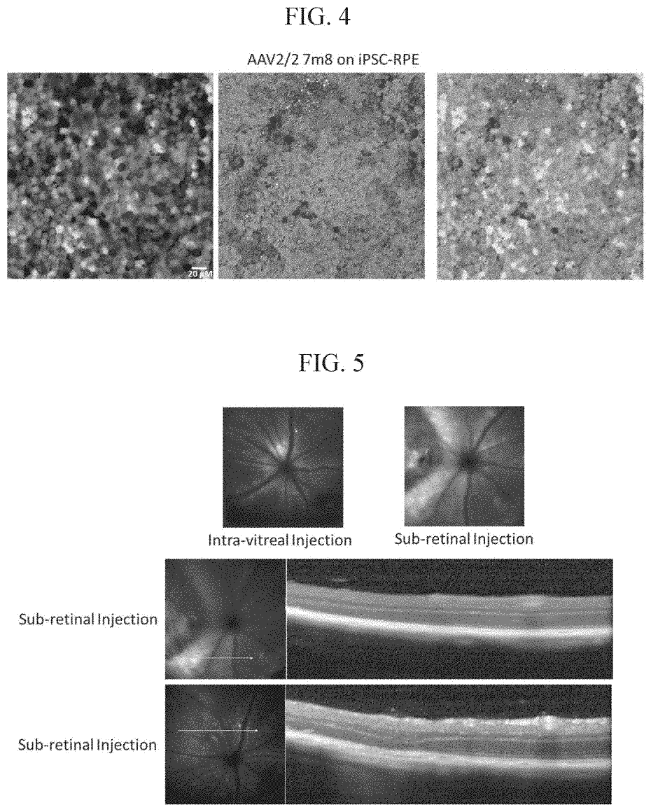

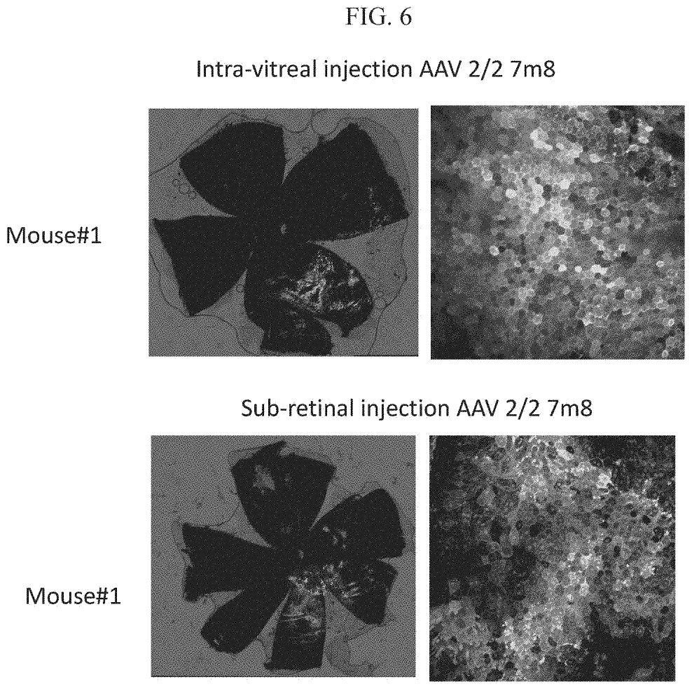

Further, intravitreal and subretinal routes were tested for gene delivery to local mouse RPE cells. FIG. 5 demonstrates that targeting and expression of the AAV vector in retina structure following injections (construct containing AAV vector of SEQ ID NO:21 was used). Specifically, the inventors demonstrate viability of gene expression in the targeted RPE cells through intravitreal injection of AAV27M8, as shown in FIG. 6.

Materials and Methods for Example 3: C57BL6 mice were obtained from our breeding facility and were maintained under a tightly controlled temperature (23 ±5° C.), humidity (40-50%) and light/dark (12/12 h) cycle conditions in a 200 lux light environment. The mice were anaesthetized by intraperitoneal injection of a ketamine (80 mg kg⁻¹), xylazine (16 mg kg⁻¹) and acepromazine (5 mg kg⁻¹) cocktail. Prior to the Intravitreal/subretinal injections, the cornea was anaesthetized with a drop of 0.5% proparacaine HCl and the pupil was dilated with 1.0% tropicamide ophthalmic solution (Bausch & Lomb Inc.). Thermal stability was maintained by placing mice on a temperature-regulated heating pad during the injection procedure and for recovery purposes. All the surgical manipulations were carried out under a surgical microscope (AmScope). A solution (2 μl) that contained 1×10\hat{ }14 viral genome, was injected. We used UMP3 ultramicropump fitted with a NanoFil syringe and the RPE-KIT (all from World Precision Instruments) equipped with a 34-gauge beveled needle.

The mice were euthanized and eyes were collected 8 days after injection. Enucleated eyes from these mice were rinsed twice with PBS, a puncture was made at ora serrata with an 18-gauge needle and the eyes were opened along the corneal incisions. The lens was then carefully removed. The eye cup was flattened, making incisions radially to the center, to give the final ‘leaf-let’ appearance. The retina was then separated gently from the RPE layer. The separated RPE layer were flat mounted on the cover-glass slide and imaged with NIS-Elements using a Nikon C2 confocal microscope (Nikon Instruments Inc.). A diode laser (488 nm) for green excitation was used to evaluate the gene expression in the RPE layer and images were captured by Low Noise PMT C2 detectors in a Plan Apo VC×20/0.75, 1 mm WD lens.

After determining the ability to target specifically RPE cells, the construction was altered to only express Kir7.1 and no GFP for therapeutic use. This vector is depicted in FIG. 7 depicts a plasmid map of the entire construct and entre sequence-verified through sequencing from ITR to ITR of the AAV vector encoding Kir7.1 to be used for gene therapy applications (FIG. 8).

FIGS. 9 and 10 show expression of KCNJ13 gene in mouse RPE cells in culture after transduction of 10\hat{ }8, 10\hat{ }9, and 10\hat{ }10 particles. A dose-dependent increase in the expression of both human KCNJ13 (FIG. 9) and AAV ITR (FIG. 10) indicates gene expression specifically within RPE cells.

1. An adeno-associated viral (AAV) gene therapy vector comprising:
a 5′ inverted terminal repeat (ITR) comprising SEQ ID NO:23,
a retinal pigment epithelium (RPE) specific promoter,
a polynucleotide that encodes a Kir7.1KCNJ13 protein and has at least 90% sequence identity to SEQ ID NO:25,
a posttranscriptional regulatory element (PRE),
a polyadenylation signal, and
a 3′_ITR of SEQ ID NO:28.

2. The AAV gene therapy vector of claim 1, wherein the RPE specific promoter is a VMD2 promoter comprising a polynucleotide having at least 90% sequence identity to SEQ ID NO:24.

3. The AAV gene therapy vector of claim 1, wherein the posttranscriptional regulatory element is a woodchuck PRE comprising SEQ ID NO:26.

4. The AAV gene therapy vector of claim 1, wherein the polyadenylation signal comprises SEQ ID NO:27.

5. The AAV gene therapy vector of claim 1, wherein the vector comprises a polynucleotide having at least 90% sequence identity to SEQ ID NO:31.

6. (canceled)

7. A construct comprising the AAV gene therapy vector of claim 1.

8. The construct of claim 7, wherein the construct is a plasmid that comprises an antibiotic resistance gene and an origin of replication and is capable of propagation in bacteria.

9. The construct of claim 7, the construct comprising a polynucleotide having at least 90% sequence identity to SEQ ID NO:22.

10. A cell comprising the construct of claim 7, wherein the cell is capable of producing AAV virus particles comprising the AAV gene therapy vector and is capable of expressing the Kir7.1 protein.

11. The cell of claim 10, wherein the cell further comprises helper plasmids that encode AAV proteins required to produce AAV virus particles.

12. (canceled)

13. An AAV virus particle made by the cell of claim 10.

14. A therapeutic composition comprising the AAV gene therapy vector of claim 1 and a pharmaceutically-acceptable carrier.

15. A method of treating a subject having a condition associated with insufficient expression or function of a Kir7.1 protein, the method comprising administering a therapeutically effective amount of the therapeutic composition of claim 14 to the subject.

16. The method of claim 15, wherein the condition is associated with at least one loss-of-function mutation in a KCNJ13 gene that results in a substitution to SEQ ID NO:1 selected from the group consisting of W53Ter, Q116R, 1120T, T1531, R162Q, R166Ter, L241P, E276A, S105I, and G219Ter within the subject.

17. (canceled)

18. The method of claim 15, wherein the condition is selected from the group consisting of Leber congenital amaurosis 16 (LCA16), retinitis pigmentosa, and snowflake vitreoretinal degeneration (SVD).

19. The method of claim 15, wherein the therapeutic composition is administered intraocularly, subretinally to at least one eye of the subject, or intravitreally to at least one eye of the subject.

20. (canceled)

21. (canceled)

22. The method of claim 15, wherein between 10⁹ and 10¹² copies of the AAV gene therapy vector are administered to the subject.

23. (canceled)

24. A method of expressing a Kir7.1 protein in a retinal pigment epithelium (RPE) cell comprising contacting the RPE cell with the adeno-associated viral vector of claim 1 in an amount effective to express the Kir7.1 protein in the RPE cell.

25. The method of claim 24, wherein the RPE cell is in vivo in a subject.

26. The method of claim 24, wherein the method is used to treat age-related macular degeneration.

27. The method of claim 24, wherein the RPE cell is ex vivo and is transplanted into a subject in need thereof.

28. (canceled)


SEQUENCE LISTING (31 sequences)

SEQ ID NO: 1 - protein, 360 aa, Homo sapiens
MDSSNCKVIAPLLSQRYRRMVTKDGHSTLQMDGAQRGLAYLRDAWGILMDMRWRWMMLVFSASFVVHWLVFAVLWYVLAEMNGDLELDHDAPPENHTICVKYITSFTAAFSFSLETQLTIGYGTMFPSGDCPSAIALLAIQMLLGLMLEAFITGAFVAKIARPKNRAFSIRFTDTAVVAHMDGKPNLIFQVANTRPSPLTSVRVSAVLYQERENGKLYQTSVDFHLDGISSDECPFFIFPLTYYHSITPSSPLATLLQHENPSHFELVVFLSAMQEGTGEICQRRTSYLPSEIMLHHCFASLLTRGSKGEYQIKMENFDKTVPEFPTPLVSKSPNRTDLDIHINGQSIDNFQISETGLTE

SEQ ID NO: 2 - DNA, 1083 bp, Homo sapiens
atggacagcagtaattgcaaagttattgctcctctcctaagtcaaagataccggaggatggtcaccaaggatggccacagcacacttcaaatggatggcgctcaaagaggtcttgcatatcttcgagatgcttggggaatcctaatggacatgcgctggcgttggatgatgttggtcttttctgcttcttttgttgtccactggcttgtctttgcagtgctctggtatgttctggctgagatgaatggtgatctggaactagatcatgatgccccacctgaaaaccacactatctgtgtcaagtatatcaccagtttcacagctgcattctccttctccctggagacacaactcacaattggttatggtaccatgttccccagtggtgactgtccaagtgcaatcgccttacttgccatacaaatgctcctaggcctcatgctagaggcttttatcacaggtgcttttgtggcgaagattgcccggccaaaaaatcgagctttttcaattcgctttactgacatagcagtagtagctcacatggatggcaaacctaatcttatcttccaagtggccaacacccgacctagccctctaaccagtgtccgggtctcagctgtactctatcaggaaagagaaaatggcaaactctaccagaccagtgtggatttccaccttgatggcatcagttctgacgaatgtccattcttcatctttccactaacgtactatcactccattacaccatcaagtcctctggctactctgctccagcatgaaaatccttctcactttgaattagttgtattcctttcagcaatgcaggagggcactggagaaatatgccaaaggaggacatcctacctacagtctgaaatcatgttacatcactgttttgcatctctgttgacccgaggttccaaatgtgaatatcaaatcaagatggagaattttgacaagactgtccctgaatttccaactcctctggtttctaaaagcccaaacaggactgacctggatatccacatcaatggacaaagcattgacaattttcagatctctgaaacaggactgacagaataa

SEQ ID NO: 3 - DNA, 1179 bp, Homo sapiens
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 4 - DNA, 626 bp, Homo sapiens
aattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccaggtcgac

SEQ ID NO: 5 - DNA, 3048 bp, Artificial Sequence
  Synthetic - EF1a-Kir7.1 DNA sequence
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgacagcagtaattgcaaagttattgctcctctcctaagtcaaagataccggaggatggtcaccaaggatggccacagcacacttcaaatggatggcgctcaaagaggtcttgcatatcttcgagatgcttggggaatcctaatggacatgcgctggcgttggatgatgttggtcttttctgcttcttttgttgtccactggcttgtctttgcagtgctctggtatgttctggctgagatgaatggtgatctggaactagatcatgatgccccacctgaaaaccacactatctgtgtcaagtatatcaccagtttcacagctgcattctccttctccctggagacacaactcacaattggttatggtaccatgttccccagtggtgactgtccaagtgcaatcgccttacttgccatacaaatgctcctaggcctcatgctagaggcttttatcacaggtgcttttgtggcgaagattgcccggccaaaaaatcgagctttttcaattcgctttactgacacagcagtagtagctcacatggatggcaaacctaatcttatcttccaagtggccaacacccgacctagccctctaaccagtgtccgggtctcagctgtactctatcaggaaagagaaaatggcaaactctaccagaccagtgtggatttccaccttgatggcatcagttctgacgaatgtccattcttcatctttccactaacgtactatcactccattacaccatcaagtcctctggctactctgctccagcatgaaaatccttctcactttgaattagttgtattcctttcagcaatgcaggagggcactggagaaatatgccaaaggaggacatcctacctaccgtctgaaatcatgttacatcactgttttgcatctctgttgacccgaggttccaaaggtgaatatcaaatcaagatggagaattttgacaagactgtccctgaatttccaactcctctggtttctaaaagcccaaacaggactgacctggatatccacatcaatggacaaagcattgacaattttcagatctctgaaacaggactgacagaataaggatcc

SEQ ID NO: 6 - DNA, 2495 bp, Artificial Sequence
  Synthetic - VMD2-Kir7.1 DNA sequence
aattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccaggtcgaccaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgacagcagtaattgcaaagttattgctcctctcctaagtcaaagataccggaggatggtcaccaaggatggccacagcacacttcaaatggatggcgctcaaagaggtcttgcatatcttcgagatgcttggggaatcctaatggacatgcgctggcgttggatgatgttggtcttttctgcttcttttgttgtccactggcttgtctttgcagtgctctggtatgttctggctgagatgaatggtgatctggaactagatcatgatgccccacctgaaaaccacactatctgtgtcaagtatatcaccagtttcacagctgcattctccttctccctggagacacaactcacaattggttatggtaccatgttccccagtggtgactgtccaagtgcaatcgccttacttgccatacaaatgctcctaggcctcatgctagaggcttttatcacaggtgcttttgtggcgaagattgcccggccaaaaaatcgagctttttcaattcgctttactgacacagcagtagtagctcacatggatggcaaacctaatcttatcttccaagtggccaacacccgacctagccctctaaccagtgtccgggtctcagctgtactctatcaggaaagagaaaatggcaaactctaccagaccagtgtggatttccaccttgatggcatcagttctgacgaatgtccattcttcatctttccactaacgtactatcactccattacaccatcaagtcctctggctactctgctccagcatgaaaatccttctcactttgaattagttgtattcctttcagcaatgcaggagggcactggagaaatatgccaaaggaggacatcctacctaccgtctgaaatcatgttacatcactgttttgcatctctgttgacccgaggttccaaaggtgaatatcaaatcaagatggagaattttgacaagactgtccctgaatttccaactcctctggtttctaaaagcccaaacaggactgacctggatatccacatcaatggacaaagcattgacaattttcagatctctgaaacaggactgacagaataaggatcc

SEQ ID NO: 7 - DNA, 26 bp, Artificial Sequence
  Synthetic - primer
gcttcgaattccgacagcagtaattg

SEQ ID NO: 8 - DNA, 26 bp, Artificial Sequence
  Synthetic - primer
atccggtggatccttattctgtcagt

SEQ ID NO: 9 - DNA, 6752 bp, Artificial Sequence
  Synthetic - AAV Viral Vector with Kir7.1
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgacagcagtaattgcaaagttattgctcctctcctaagtcaaagataccggaggatggtcaccaaggatggccacagcacacttcaaatggatggcgctcaaagaggtcttgcatatcttcgagatgcttggggaatcctaatggacatgcgctggcgttggatgatgttggtcttttctgcttcttttgttgtccactggcttgtctttgcagtgctctggtatgttctggctgagatgaatggtgatctggaactagatcatgatgccccacctgaaaaccacactatctgtgtcaagtatatcaccagtttcacagctgcattctccttctccctggagacacaactcacaattggttatggtaccatgttccccagtggtgactgtccaagtgcaatcgccttacttgccatacaaatgctcctaggcctcatgctagaggcttttatcacaggtgcttttgtggcgaagattgcccggccaaaaaatcgagctttttcaattcgctttactgacacagcagtagtagctcacatggatggcaaacctaatcttatcttccaagtggccaacacccgacctagccctctaaccagtgtccgggtctcagctgtactctatcaggaaagagaaaatggcaaactctaccagaccagtgtggatttccaccttgatggcatcagttctgacgaatgtccattcttcatctttccactaacgtactatcactccattacaccatcaagtcctctggctactctgctccagcatgaaaatccttctcactttgaattagttgtattcctttcagcaatgcaggagggcactggagaaatatgccaaaggaggacatcctacctaccgtctgaaatcatgttacatcactgttttgcatctctgttgacccgaggttccaaaggtgaatatcaaatcaagatggagaattttgacaagactgtccctgaatttccaactcctctggtttctaaaagcccaaacaggactgacctggatatccacatcaatggacaaagcattgacaattttcagatctctgaaacaggactgacagaataaggatccacccagctttcttgtacaaagtggtgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaatcgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattg

SEQ ID NO: 10 - DNA, 6752 bp, Artificial Sequence; identical to SEQ ID NO: 9
  Synthetic - Lentivirus Viral Vector with Kir7.1

SEQ ID NO: 11 - DNA, 31 bp, Artificial Sequence
  Synthetic - primer
ccgctcgagtaccttccaagtgctgtcaaac

SEQ ID NO: 12 - DNA, 30 bp, Artificial Sequence
  Synthetic - primer
cgacgcgtcatgctgaattccttaatttgc

SEQ ID NO: 13 - DNA, 30 bp, Artificial Sequence
  Synthetic - primer
ctagctagctcctcccagcgtaacgtgagc

SEQ ID NO: 14 - DNA, 28 bp, Artificial Sequence
  Synthetic - primer
gaagatctctagtggcagccccatggtg

SEQ ID NO: 15 - DNA, 32 bp, Artificial Sequence
  Synthetic - primer
ctagctagcctgtcctcttaggcagatacaga

SEQ ID NO: 16 - DNA, 30 bp, Artificial Sequence
  Synthetic - primer
gaagatctagagccttcatgttgactgcta

SEQ ID NO: 17 - DNA, 20 bp, Artificial Sequence
  Synthetic - primer
gaaagttggggatgaggcga

SEQ ID NO: 18 - DNA, 33 bp, Artificial Sequence
  Synthetic - primer
caatcaaagcttcctcagagctgccgggcggct

SEQ ID NO: 19 - DNA, 21 bp, Artificial Sequence
  Synthetic - normal TGG sequence
gacatgcgctggcgttggatg

SEQ ID NO: 20 - DNA, 21 bp, Artificial Sequence
  Synthetic - TAG sequence
gacatgcgctagcgttggatg

SEQ ID NO: 21 - DNA, 6594 bp, Artificial Sequence
  Synthetic- VB200106-1361jvy vector
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttggagcccttgagtatggattgatgtattaaaatttattgaatcacatgctgagattttcaccagctgcccgtggggatctgggcatttattcccatattgcactggctggctggaagccagcagcataaactccagggctgttctgtcaacccccaccagactcacccccctccaccagccccggcaggcttctccttccatctctctgaagcaacttactgatgggccctgccagccaatcacagccagaataacgtatgatgtcaccagcagccaatcagagctcctcgtcagcatatgcagaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagaccttctgtgggatcatcggacccacctggaaccccacctgtgagtacaaggtcaagtttgtacaaaaaagcaggctgccaccaagcttatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggacagcagtaattgcaaagttattgctcctctcctaagtcaaagataccggaggatggtcaccaaggatggccacagcacacttcaaatggatggcgctcaaagaggtcttgcatatcttcgagatgcttggggaatcctaatggacatgcgctggcgttggatgatgttggtcttttctgcttcttttgttgtccactggcttgtctttgcagtgctctggtatgttctggctgagatgaatggtgatctggaactagatcatgatgccccacctgaaaaccacactatctgtgtcaagtatatcaccagtttcacagctgcattctccttctccctggagacacaactcacaattggttatggtaccatgttccccagtggtgactgtccaagtgcaatcgccttacttgccatacaaatgctcctaggcctcatgctagaggcttttatcacaggtgcttttgtggcgaagattgcccggccaaaaaatcgagctttttcaattcgctttactgacacagcagtagtagctcacatggatggcaaacctaatcttatcttccaagtggccaacacccgacctagccctctaaccagtgtccgggtctcagctgtactctatcaggaaagagaaaatggcaaactctaccagaccagtgtggatttccaccttgatggcatcagttctgacgaatgtccattcttcatctttccactaacgtactatcactccattacaccatcaagtcctctggctactctgctccagcatgaaaatccttctcactttgaattagttgtattcctttcagcaatgcaggagggcactggagaaatatgccaaaggaggacatcctacctaccgtctgaaatcatgttacatcactgttttgcatctctgttgacccgaggttccaaaggtgaatatcaaatcaagatggagaattttgacaagactgtccctgaatttccaactcctctggtttctaaaagcccaaacaggactgacctggatatccacatcaatggacaaagcattgacaattttcagatctctgaaacaggactgacagaataaggatccacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 22 - DNA, 5880 bp, Artificial Sequence
  Synthetic - AAV-VMD2-Kir7.1 vector
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttggagcccttgagtatggattgatgtattaaaatttattgaatcacatgctgagattttcaccagctgcccgtggggatctgggcatttattcccatattgcactggctggctggaagccagcagcataaactccagggctgttctgtcaacccccaccagactcacccccctccaccagccccggcaggcttctccttccatctctctgaagcaacttactgatgggccctgccagccaatcacagccagaataacgtatgatgtcaccagcagccaatcagagctcctcgtcagcatatgcagaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagaccttctgtgggatcatcggacccacctggaaccccacctgtgagtacaaggtcaagtttgtacaaaaaagcaggctgccaccaagcttatggacagcagtaattgcaaagttattgctcctctcctaagtcaaagataccggaggatggtcaccaaggatggccacagcacacttcaaatggatggcgctcaaagaggtcttgcatatcttcgagatgcttggggaatcctaatggacatgcgctggcgttggatgatgttggtcttttctgcttcttttgttgtccactggcttgtctttgcagtgctctggtatgttctggctgagatgaatggtgatctggaactagatcatgatgccccacctgaaaaccacactatctgtgtcaagtatatcaccagtttcacagctgcattctccttctccctggagacacaactcacaattggttatggtaccatgttccccagtggtgactgtccaagtgcaatcgccttacttgccatacaaatgctcctaggcctcatgctagaggcttttatcacaggtgcttttgtggcgaagattgcccggccaaaaaatcgagctttttcaattcgctttactgacacagcagtagtagctcacatggatggcaaacctaatcttatcttccaagtggccaacacccgacctagccctctaaccagtgtccgggtctcagctgtactctatcaggaaagagaaaatggcaaactctaccagaccagtgtggatttccaccttgatggcatcagttctgacgaatgtccattcttcatctttccactaacgtactatcactccattacaccatcaagtcctctggctactctgctccagcatgaaaatccttctcactttgaattagttgtattcctttcagcaatgcaggagggcactggagaaatatgccaaaggaggacatcctacctaccgtctgaaatcatgttacatcactgttttgcatctctgttgacccgaggttccaaaggtgaatatcaaatcaagatggagaattttgacaagactgtccctgaatttccaactcctctggtttctaaaagcccaaacaggactgacctggatatccacatcaatggacaaagcattgacaattttcagatctctgaaacaggactgacagaataaggatccacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 23 - DNA, 141 bp, Artificial Sequence
  Synthetic - 5′ITR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 24 - DNA, 976 bp, Artificial Sequence
  Synthetic - VMD2 promoter
gagcccttgagtatggattgatgtattaaaatttattgaatcacatgctgagattttcaccagctgcccgtggggatctgggcatttattcccatattgcactggctggctggaagccagcagcataaactccagggctgttctgtcaacccccaccagactcacccccctccaccagccccggcaggcttctccttccatctctctgaagcaacttactgatgggccctgccagccaatcacagccagaataacgtatgatgtcaccagcagccaatcagagctcctcgtcagcatatgcagaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagaccttctgtgggatcatcggacccacctggaaccccacctgtgagtacaaggt

SEQ ID NO: 25 - DNA, 1094 bp, Artificial Sequence
  Synthetic - KCJN13
ccaccaagcttatggacagcagtaattgcaaagttattgctcctctcctaagtcaaagataccggaggatggtcaccaaggatggccacagcacacttcaaatggatggcgctcaaagaggtcttgcatatcttcgagatgcttggggaatcctaatggacatgcgctggcgttggatgatgttggtcttttctgcttcttttgttgtccactggcttgtctttgcagtgctctggtatgttctggctgagatgaatggtgatctggaactagatcatgatgccccacctgaaaaccacactatctgtgtcaagtatatcaccagtttcacagctgcattctccttctccctggagacacaactcacaattggttatggtaccatgttccccagtggtgactgtccaagtgcaatcgccttacttgccatacaaatgctcctaggcctcatgctagaggcttttatcacaggtgcttttgtggcgaagattgcccggccaaaaaatcgagctttttcaattcgctttactgacacagcagtagtagctcacatggatggcaaacctaatcttatcttccaagtggccaacacccgacctagccctctaaccagtgtccgggtctcagctgtactctatcaggaaagagaaaatggcaaactctaccagaccagtgtggatttccaccttgatggcatcagttctgacgaatgtccattcttcatctttccactaacgtactatcactccattacaccatcaagtcctctggctactctgctccagcatgaaaatccttctcactttgaattagttgtattcctttcagcaatgcaggagggcactggagaaatatgccaaaggaggacatcctacctaccgtctgaaatcatgttacatcactgttttgcatctctgttgacccgaggttccaaaggtgaatatcaaatcaagatggagaattttgacaagactgtccctgaatttccaactcctctggtttctaaaagcccaaacaggactgacctggatatccacatcaatggacaaagcattgacaattttcagatctctgaaacaggactgacagaataa

SEQ ID NO: 26 - DNA, 598 bp, Artificial Sequence
  Synthetic - woodchuck post translational regulatory element
cgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgg

SEQ ID NO: 27 - DNA, 208 bp, Artificial Sequence
  Synthetic - BGHpA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 28 - DNA, 141 bp, Artificial Sequence
  Synthetic - 3′ ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 29 - DNA, 861 bp, Artificial Sequence
  Synthetic - ampicillin drug resistance gene
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 30 - DNA, 589 bp, Artificial Sequence
  Synthetic - pUC ori
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 31 - DNA, 3283 bp, Artificial Sequence
  Synthetic - Packaged sequence from the 5′ITR to the end of the 3′ITR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttggagcccttgagtatggattgatgtattaaaatttattgaatcacatgctgagattttcaccagctgcccgtggggatctgggcatttattcccatattgcactggctggctggaagccagcagcataaactccagggctgttctgtcaacccccaccagactcacccccctccaccagccccggcaggcttctccttccatctctctgaagcaacttactgatgggccctgccagccaatcacagccagaataacgtatgatgtcaccagcagccaatcagagctcctcgtcagcatatgcagaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagaccttctgtgggatcatcggacccacctggaaccccacctgtgagtacaaggtcaagtttgtacaaaaaagcaggctgccaccaagcttatggacagcagtaattgcaaagttattgctcctctcctaagtcaaagataccggaggatggtcaccaaggatggccacagcacacttcaaatggatggcgctcaaagaggtcttgcatatcttcgagatgcttggggaatcctaatggacatgcgctggcgttggatgatgttggtcttttctgcttcttttgttgtccactggcttgtctttgcagtgctctggtatgttctggctgagatgaatggtgatctggaactagatcatgatgccccacctgaaaaccacactatctgtgtcaagtatatcaccagtttcacagctgcattctccttctccctggagacacaactcacaattggttatggtaccatgttccccagtggtgactgtccaagtgcaatcgccttacttgccatacaaatgctcctaggcctcatgctagaggcttttatcacaggtgcttttgtggcgaagattgcccggccaaaaaatcgagctttttcaattcgctttactgacacagcagtagtagctcacatggatggcaaacctaatcttatcttccaagtggccaacacccgacctagccctctaaccagtgtccgggtctcagctgtactctatcaggaaagagaaaatggcaaactctaccagaccagtgtggatttccaccttgatggcatcagttctgacgaatgtccattcttcatctttccactaacgtactatcactccattacaccatcaagtcctctggctactctgctccagcatgaaaatccttctcactttgaattagttgtattcctttcagcaatgcaggagggcactggagaaatatgccaaaggaggacatcctacctaccgtctgaaatcatgttacatcactgttttgcatctctgttgacccgaggttccaaaggtgaatatcaaatcaagatggagaattttgacaagactgtccctgaatttccaactcctctggtttctaaaagcccaaacaggactgacctggatatccacatcaatggacaaagcattgacaattttcagatctctgaaacaggactgacagaataaggatccacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg